(19) United States
(12) Patent Application Publication
(10) Pub. No.: US 2022/0096548 A1
(43) Pub. Date: Mar. 31, 2022

(54) MODIFIED IMMUNE CELLS CO-EXPRESSING CHIMERIC ANTIGEN RECEPTOR AND IL-6 ANTAGONIST FOR REDUCING TOXICITY AND USES THEREOF IN ADOPTIVE CELL THERAPY

(71) Applicant: Hunan Siweikang Therapeutics Co. Ltd., Changsha Hunan (CN)

(72) Inventor: Biliang HU, Worcester, MA (US)

(21) Appl. No.: 17/420,967

(22) PCT Filed: Jan. 6, 2020
(86) PCT No.: PCT/US2020/012329

§ 371 Date: Jul. 6, 2021

Related U.S. Application Data
(60) Provisional application No. 62/928,720, filed on Oct. 31, 2019.
(60) Provisional application No. 62/855,250, filed on May 31, 2019.
(60) Provisional application No. 62/789,311, filed on Jan. 7, 2019.

(51) Int. Cl. A61K 35/17; C07K 16/24; C07K 16/28; A61P 35/00; C07K 16/46

(52) U.S. Cl. CPC A61K 35/17; C07K 16/248; C07K 16/468; A61P 35/00; C07K 16/2878; C07K 16/2866

(57) ABSTRACT

A population of immune cells comprising modified immune cells co-expressing a chimeric antigen receptor and an IL-6 signaling antagonist (e.g., an anti-IL6 or anti-IL-6R antibody) and optionally an IL-1 signaling antagonist. Also provided herein are methods of producing such immune cell populations comprising the modified immune cells and methods of using such in cell therapy (e.g., to treat cancer, infectious diseases, or immune diseases).

### CROSS REFERENCE TO RELATED APPLICATIONS

This application claims the benefit of the filing dates of U.S. Provisional Application No. 62/789,311, filed Jan. 7, 2019, U.S. Provisional Application No. 62/855,250, filed May 31, 2019, and U.S. Provisional Application No. 62/928,720, filed Oct. 31, 2019, the entire contents of each of which is incorporated by reference herein.

### BACKGROUND OF THE INVENTION

Adoptive cell transfer therapy is a type of immunotherapy that involves ex vivo expansion of autologous or allogeneic immune cells and subsequent infusion into a patient. The immune cells may be modified ex vivo to specifically target malignant cells. The promise of adoptive cell transfer therapy is often limited by toxicity (e.g., cytokine-associated toxicity). For example, adoptive cell transfer immunotherapy may trigger non-physiologic elevation of cytokine levels (cytokine release syndrome), which could lead to death of recipients (see, e.g., Morgan et al., Molecular Therapy 18(4): 843-851, 2010).

It is therefore of great interest to develop approaches to reduce toxicity associated with adoptive cell transfer immunotherapy, while maintaining efficacy.

### SUMMARY OF THE INVENTION

The present disclosure is based on the discovery that certain antibodies targeting interleukin 6 (IL-6) or interleukin 6 receptor (IL-6R) showed unexpectedly higher efficacy in inhibiting the IL-6 signaling pathway as relative to other IL-6 antagonists. Such anti-IL-6 or anti-IL-6R would be expected to be more effective in reducing IL-6-mediated toxicity in CAR-T therapy.

Accordingly, one aspect of the instant disclosure provides a population of immune cells, wherein the immune cells express a chimeric receptor antigen (CAR) and an antibody specific to interleukin-6 (IL-6) or IL-6 receptor (IL-6R), wherein the antibody comprises the same heavy chain complementarity determining domains (CDRs) and the same light chain CDRs as a reference antibody, and wherein the reference antibody comprises (a) a heavy chain variable domain (V_{H}) set forth as SEQ ID NO: 1 and a light chain variable domain (V_{L}) set forth as SEQ ID NO: 2, or (b) a V_{H} set forth as SEQ ID NO: 3 and a V_{L} set forth as SEQ ID NO: 4. In some instances, the antibody specific to IL-6 or IL-6R comprises the same V_{H} and the same V_{L} as the reference antibody. Such antibodies may be a single-chain antibody fragment (scFv). In specific examples, the scFv may comprise the amino acid sequence of SEQ ID NO: 13 or SEQ ID NO: 14.

In some embodiments, the population of immune cells as described herein may contain at least 10% (e.g., 10%-90% or 50%-70% or more) of the cells that express both the CAR and the antibody specific to IL-6 or IL-6R.

In some embodiments, the population of immune cells described herein may be T-cells, NK cells, dendritic cells, macrophages, B cells, neutrophils, eosinophils, basophils, mast cells, myeloid-derived suppressor cells, mesenchymal stem cells, precursors thereof, or a combination thereof. In some instances, the immune cells are T cells, which do not express an endogenous T cell receptor.

In some embodiments, the population of immune cells expresses a CAR, which comprises an extracellular domain specific to a pathologic antigen, a transmembrane domain, and a cytoplasmic domain comprising one or more signaling domains. The one or more signaling domains may comprise one or more co-stimulatory domains, CD3ζ, or a combination thereof. Exemplary co-stimulatory domains include, but are not limited to those from CD28, 4-1BB, CD27, OX40, and ICOS.

In another aspect, provided herein is an immune cell, which expresses a chimeric receptor antigen (CAR) and an antibody specific to interleukin-6 (IL-6) or IL-6 receptor (IL-6R), wherein the antibody comprises the same heavy chain complementarity determining domains (CDRs) and the same light chain CDRs as a reference antibody, and wherein the reference antibody comprises (a) a heavy chain variable domain (V_{H}) set forth as SEQ ID NO: 1 and a light chain variable domain (V_{L}) set forth as SEQ ID NO: 2, or (b) a V_{H} set forth as SEQ ID NO: 3 and a V_{L} set forth as SEQ ID NO: 4. In some instances, the antibody specific to IL-6 or IL-6R may comprise the same V_{H} and the same V_{L} as the reference antibody. Such antibodies may be single-chain antibody fragments (scFv). In specific examples, the scFv may comprise the amino acid sequence of SEQ ID NO: 13 or SEQ ID NO: 14.

In any or the immune cells or immune cell populations described herein, the antibody specific to interleukin-6 (IL-6) or IL-6 receptor (IL-6R) is a fragment of a bi-specific antibody, which further comprises an antibody specific to granulocyte macrophage-colony stimulating factor (GM-CSF). The antibody specific to GM-CSF comprises the same heavy chain complementarity determining domains (CDRs) and the same light chain CDRs as a reference antibody, and wherein the reference antibody comprises (a) a heavy chain variable domain (V_{H}) set forth as SEQ ID NO: 21 and a light chain variable domain (V_{L}) set forth as SEQ ID NO: 22. In some examples, the antibody specific to GM-CSF comprises a V_{H} set forth as SEQ ID NO: 21 and a V_{L} set forth as SEQ ID NO: 22.

In some examples, the antibody specific to GM-CSF is a scFv antibody. Such a scFv antibody maybe linked to the antibody specific to IL-6 or IL-6R via a peptide linker, e.g., GSGGSG. In one specific example, the bispecific antibody comprises the amino acid sequence of SEQ ID NO: 28.

Alternatively or in addition, any of the immune cells or immune cell populations described herein may express an IL-1 antagonist. In some examples, the IL-1 antagonist is IL1RA, which may comprise the amino acid sequence of SEQ ID NO: 29.

Further, any of the immune cells or immune cell populations described herein may comprise a disrupted endogenous IL-2, a disrupted endogenous GM-CSF, a disrupted endogenous TNFA, a disrupted endogenous T-cell receptor (TCR) gene, or a combination thereof.

In specific examples, the immune cell(s) described herein express an anti-CD19 CAR (e.g., a CAR comprising an extracellular antigen binding domain that comprises SEQ ID NO:52), an IL-6 antagonist (e.g., an scFv comprising SEQ ID NO:13 or SEQ ID NO:14), an IL-1 antagonist (e.g., IL-1RA). Such an immune cell(s) may have disrupted endogenous GM-CSF and/or TCR genes. In some instances, both endogenous GM-CSF and TCR genes are disrupted in the genetically engineered immune cells disclosed herein. Alternatively, such an immune cell(s) may have wild-type endogenous GM-CSF and/or TCR genes. In some instances, the immunes may have both wild-type endogenous GM-CSF and wild-type endogenous TCR genes.

In specific examples, the immune cell(s) described herein express an anti-BCMA CAR (e.g., a CAR comprising an extracellular antigen binding domain that comprises SEQ ID NO:57), an IL-6 antagonist (e.g., an scFv comprising SEQ ID NO:13 or SEQ ID NO:14), an IL-1 antagonist (e.g., IL-1RA). Such an immune cell(s) may have disrupted endogenous GM-CSF and/or TCR genes. In some instances, both endogenous GM-CSF and TCR genes are disrupted in the genetically engineered immune cells disclosed herein. Alternatively, such an immune cell(s) may have wild-type endogenous GM-CSF and TCR genes. In some instances, the immunes may have both wild-type endogenous GM-CSF and wild-type endogenous TCR genes.

Any of the bi-specific antibodies capable of binding to both IL-6/IL-6R and GM-CSF described herein is also within the scope of the present disclosure.

Further, the present disclosure provides a nucleic acid comprising a first nucleotide sequence encoding an antibody fragment specific to IL-6, a second nucleotide sequence encoding a self-cleaving peptide, and a third nucleic sequence encoding an IL-1 antagonist. In some instances, the first nucleotide sequence encodes a bi-specific antibody comprising the antibody fragment specific to IL-6 and an antibody fragment specific to GM-CSF.

In yet another aspect, the present disclosure provides a method of producing a population of modified immune cells with reduced inflammatory properties, the method comprising: (i) providing a population of immune cells (e.g., those described herein); and (ii) introducing into the immune cells a first nucleic acid coding for a chimeric antigen receptor (CAR) as described herein and a second nucleic acid coding for an antibody specific to interleukin-6 (IL-6) or IL-6 receptor (IL-6R) as also described herein. Both the first nucleic acid and the second nucleic acid are in operable linkage to a promoter for expression of the CAR and the antibody in the immune cells. When the immune cells are T cells, the method may further comprise disrupting an endogenous T cell receptor gene.

Further, the present disclosure provides a method of cell therapy, comprising administering to a subject in need thereof the population of immune cells or the immune cell as described herein. In some instances, the subject is a human patient having cancer, an infectious disease, or an immune disorder. In some instances, the subject is a human patient having a cancer, and wherein the human patient has subjected to a therapy against the cancer to reduce tumor burden. Exemplary anti-cancer therapies include, but are not limited to, chemotherapy, immunotherapy, radiotherapy, surgery, or a combination thereof. In some embodiments, the subject may be treated by a conditioning regimen after the anti-cancer therapy to deplete endogenous lymphocytes so as to place the subject in condition for the cell therapy disclosed herein.

In some instances, the immune cells used in the method of cell therapy disclosed herein express an anti-CD19 CAR (e.g., a CAR comprising an extracellular antigen binding domain that comprises SEQ ID NO:52), an IL-6 antagonist (e.g., an scFv comprising SEQ ID NO:13 or SEQ ID NO:14), an IL-1 antagonist (e.g., IL-1RA). Such an immune cell(s) may have disrupted endogenous GM-CSF and/or TCR genes. In some instances, both endogenous GM-CSF and TCR genes are disrupted in the genetically engineered immune cells disclosed herein. Alternatively, such an immune cell(s) may have wild-type endogenous GM-CSF and/or TCR genes. In some instances, the immunes may have both wild-type endogenous GM-CSF and wild-type endogenous TCR genes. Such immune cells can be used to treat human patients having lymphoblastic leukemia (e.g., acute lymphoblastic leukemia) or non-Hodgkin lymphoma.

In some instances, the immune cells used in the method of cell therapy disclosed herein express an anti-BCMACAR (e.g., a CAR comprising an extracellular antigen binding domain that comprises SEQ ID NO:57), an IL-6 antagonist (e.g., an scFv comprising SEQ ID NO:13 or SEQ ID NO:14), an IL-1 antagonist (e.g., IL-1RA). Such an immune cell(s) may have disrupted endogenous GM-CSF and/or TCR genes. In some instances, both endogenous GM-CSF and TCR genes are disrupted in the genetically engineered immune cells disclosed herein. Alternatively, such an immune cell(s) may have wild-type endogenous GM-CSF and/or TCR genes. In some instances, the immunes may have both wild-type endogenous GM-CSF and wild-type endogenous TCR genes. Such immune cells can be used to treat human patients having multiple myeloma, such as relapsed or refractory multiple myeloma.

Also within the scope of the present disclosure are immune cell populations as described herein for use in treating the target disease as also described herein, and uses of such immune cell population in manufacturing a medicament for use in treatment of a target disease.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a chart showing the effects of inhibiting IL-6 signaling by various antibodies specific to IL-6 or IL-6R expressed by 293T cells resulting from transient transfection of a 3^{rd} generation lentiviral vector encoding T2A linked anti-CD19 CAR and a certain antibody.

FIGS. 2A-2B include charts showing the combined effects of IL-6 antagonist and IL-1 antagonist in inhibiting both the IL-1 signaling (2A) and IL-6 signaling (2B).

FIGS. 3A-3C include charts showing the effects of anti-IL-6/anti-GM-CSF bispecific antibodies in combination with IL1RA on the GM-CSF signaling (3A), IL-6 signaling (3B), and IL-1 signaling (3C).

FIG. 4A-4B includes charts showing proliferation and cytokine expression of T cell with wild-type GM-CSF and T cells having gene editing of endogenous GM-CSF via CRISPR.

FIG. 5A-5B includes charts showing proliferation and cytokine expression of wild-type T cell and T cells having genetically edited endogenous IL-2 via CRISPR.

FIG. 6A-6B includes charts showing proliferation and cytokine expression of wild-type T cell and T cells having genetically edited endogenous TNFA via CRISPR.

FIG. 7A-7D includes charts showing anti-CD19/IL6/IL1 TCR- and anti-CD19/IL6/IL1 TCR⁻/GM-CSF⁻ cells exert IL6 (7A) and IL1B (7B) inhibitory effect, secret similar levels of IFNγ, IL-2 and TNFα, while secretion of GM-CSF is significantly reduced (7C) and able to induce cytotoxicity in CD19+ Nalm6 cells (7D).

FIG. 8A-8D includes charts showing anti-BCMA/IL6/IL1GM-CSF⁻ cells exert IL6 (8A) and IL1B (8B) inhibitory effect, secret similar levels of IFNγ and IL-2, while secretion of GM-CSF is significantly reduced (8C) and able to induce cytotoxicity in BCMA⁺ RPMI-8226 cells (8D).

FIGS. 9A-9H include charts showing cytokine secretion levels and other cytokine release syndrome (CRS) features in a human cancer patient after a first round of anti-CD19/IL6/IL1/GM-CSF KO CAR-T cell treatment. FIG. 9A: Levels of GM-CSF⁺ cells in GM-CSF KO T cells and the wild-type counterpart. FIG. 9B: Levels of GM-CSF in the human patient at different time points as indicated after the T cell infusion. FIG. 9C: Levels of IL-6 in the human patient at different time points as indicated after the T cell infusion. FIG. 9D: Levels of IL1/IL1R Blocker in the human patient at different time points as indicated after the T cell infusion. FIG. 9E: Levels of CAR vector copies in the human patient at different time points as indicated after the T cell infusion. FIG. 9F: Levels of C-Reactive Protein (CRP) in the human patient at different time points as indicated after the T cell infusion. FIG. 9G: Levels of interferon γ (IFNγ) in the human patient at different time points as indicated after the T cell infusion. FIG. 9H is a chart showing the daily maximum temperature (Tmax ° C.) of the patient.

FIGS. 10A-10H include charts showing cytokine secretion levels and other cytokine release syndrome (CRS) features in the same human cancer patient after a second round of anti-CD19/IL6/IL1/CAR-T cell treatment. FIG. 10A: Levels of IL-6 in the human patient at different time points as indicated after the T cell infusion. FIG. 10B: Daily maximum body temperature (° C.) of the human patient at different time points as indicated after the T cell infusion. FIG. 10C: Levels of IL1/IL1R Blocker in the human patient at different time points as indicated after the T cell infusion. FIG. 10D: Levels of GM-CSF in the human patient at different time points as indicated after the T cell infusion. FIG. 10E: Levels of CRP in the human patient at different time points as indicated after the T cell infusion. FIG. 10F: Levels of CAR vector copies in the human patient at different time points as indicated after the T cell infusion. FIG. 10G: Numbers of CAR-T cells in the human patient at different time points as indicated after the T cell infusion. FIG. 10H: Levels of IFNγ in the human patient at different time points as indicated after the T cell infusion.

FIGS. 11A-11K include diagrams showing clinical features of a refractory multiple myeloma patient treating with anti-BCMA CAR-T cells expressing IL6 and IL-1 antagonists and having GM-CSF and TCR genes knocked out. FIG. 11A: Knockout efficiency of GM-CSF by Crispr/Cas9 gene editing during ex-vivo expansion of patient T cells. FIG. 11B: Change of IgA concentration before and after CART treatment. FIG. 11C: Concentration of IL-6 in peripheral blood during CART treatment. FIG. 11D: Change of daily temperature during CART treatment. FIG. 11E: Concentration of CRP in peripheral blood during CART treatment. FIG. 11F: Concentration of IL1/IL1R blocker in peripheral blood during CART treatment. FIG. 11G: CAR vector copies per μg genomic DNA in peripheral blood during CART treatment. FIG. 11H: Total number of CAR+ T cells in peripheral blood during CART treatment. FIG. 11I: Concentration of IFNγ in peripheral blood during CAR-T therapy. FIG. 11J: The concentration of GM-CSF in peripheral blood during CART treatment. FIG. 11K: The comparison of IFNγ and IL6 levels in peripheral blood during CART treatment.

FIGS. 12A-12D include diagrams showing therapeutic effects of anti-CD19 CAR-T cells expressing IL6 and IL-1 antagonists and having the TCR gene knocked out. Such T cells either have wild-type GM-CSF or knocked-out GM-CSF. FIG. 12A: a chart showing change of mouse body weight after treatment. FIG. 12B: a chart showing survival rate of treated mice. FIG. 12C: a chart showing change of leukemia cell level in the blood of treated mice. FIG. 12D: a chart showing change of T cell level in the blood of treated mice.

FIGS. 13A-13K include diagrams showing clinical features of a non-Hodgkin human patient treating with anti-CD19 CAR-T cells expressing IL6 and IL-1 antagonists and having GM-CSF and TCR genes knocked out. FIG. 13A: a chart showing knockout efficiency of GM-CSF by Crispr/Cas9 gene editing during ex-vivo expansion of patient T cells. FIG. 13B: a diagram showing concentration of IL-6 in peripheral blood during the CAR-T treatment. FIG. 13C: a diagram showing concentration of IFNG in peripheral blood during the CAR-T treatment. FIG. 13D: a chart comparing the concentrations of IL-6 and IFNG in peripheral blood of the patient during the CAR-T treatment. FIG. 13E: a diagram showing concentration of IL1/IL1R blocker in peripheral blood during CART treatment. FIG. 13F: a diagram showing concentration of GM-CSF in peripheral blood during CART treatment. FIG. 13G: a diagram showing copies of the CAR vector per μg of genomic DNAs in peripheral blood of the patient during the CAR-T treatment. FIG. 13H: a chart showing daily maximum body temperature (° C.) of the human patient at various time points as indicated after the T cell infusion. FIG. 13I: a diagram showing the levels of CRP in the human patient at various time points as indicated after the T cell infusion.

FIGS. 14A-14K include diagrams showing clinical features of an acute lymphoblastic leukemia human patient treating with anti-CD19 CAR-T cells expressing IL6 and IL-1 antagonists. FIG. 14A: a diagram showing concentration of IL-6 in peripheral blood of the patient at various time points as indicated after T cell infusion. FIG. 14B: a diagram showing concentration of IFNG in peripheral blood of the patient at various time points as indicated after T cell infusion. FIG. 14C: a diagram comparing concentrations of IL-6 and IFNG in peripheral blood of the patient at various time points as indicated after T cell infusion. FIG. 14D: a diagram showing concentration of the IL1/IL1R blocker in peripheral blood of the patient at various time points as indicated after T cell infusion. FIG. 14E: a diagram showing concentration of GM-CSF in peripheral blood of the patient at various time points as indicated after T cell infusion. FIG. 14F: a diagram showing change of body temperature in the human patient at various time points as indicated after T cell infusion. FIG. 14G: a diagram showing the levels of CAR vector copies in the patient at various time points as indicated after T cell infusion. FIG. 14H: a diagram showing the levels of CRP in peripheral blood of the patient at various time points as indicated after T cell infusion.

FIGS. 15A-15P include diagrams showing clinical features of a multiple myeloma patient treating with anti-BCMA CAR-T cells expressing IL6 and IL-1 antagonists. FIG. 15A: a diagram showing the change of daily body temperature in the patient at various time points after CAR-T cell infusion. FIG. 15B: a diagram showing the concentration of CRP in the peripheral blood of the patient at various time points after CAR-T cell infusion. FIG. 15C: a diagram showing the concentration of ferritin in the peripheral blood of the patient at various time points after CAR-T cell infusion. FIG. 15D: a diagram showing the levels of IFNG in the peripheral blood of the patient at various time points after CAR-T cell infusion. FIG. 15E: a diagram showing the level of IL-6 in the peripheral blood of the patient at various time points after CAR-T cell infusion. FIG. 15F: a diagram comparing the levels of IL-6 and IFNG in the peripheral blood of the patient at various time points after CAR-T cell infusion. FIG. 15G: a diagram showing the copy numbers of the CAR vector per μg of genomic DNAs in the peripheral blood of the patient at various time points after CAR-T cell infusion. FIG. 15H: a diagram showing the levels of the IL1/IL1R blocker in the peripheral blood of the patient at various time points after CAR-T cell infusion. FIG. 15I: a diagram comparing the levels of IL1RA and IL-6 in the peripheral blood of the patient at various time points after CAR-T cell infusion. FIG. 15J: a diagram showing the levels of IL-1B in the peripheral blood of the patient at various time points after CAR-T cell infusion. FIG. 15K: a diagram showing the levels of IL-2 in the peripheral blood of the patient at various time points after CAR-T cell infusion. FIG. 15L: a diagram showing the levels of IL-4 in the peripheral blood of the patient at various time points after CAR-T cell infusion. FIG. 15M: a diagram showing the levels of IL-10 in the peripheral blood of the patient at various time points after CAR-T cell infusion. FIG. 15N: a diagram showing the levels of IL-17A in the peripheral blood of the patient at various time points after CAR-T cell infusion. FIG. 15O: a diagram showing the levels of TNFA in the peripheral blood of the patient at various time points after CAR-T cell infusion. FIG. 15P: a diagram showing the levels of GM-CSF in the peripheral blood of the patient at various time points after CAR-T cell infusion.

### DETAILED DESCRIPTION OF THE INVENTION

Adoptive cell transfer immunotherapy relies on immune cell activation and cytokine secretion to eliminate disease cells. However, systemic overproduction of cytokines raises safety concerns and sometimes can be fatal to the recipients. Morgan et al., Molecular Therapy 18(4):843-851, 2010. The present disclosure aims to overcome this limitation, in part, via the development of immune cells having reduced inflammatory properties. Cytokine release syndrome (CRS) is a common type of toxicity associated with CAR-T cell therapy. Tocilizumab, an anti-IL6R antibody, is commonly used for alleviating CRS in CAR-T therapy. However, a high level of IL-6 would still circulate in a patient who develops CRS after a CAR-T therapy. Such IL-6 molecules could pass through the blood brain barrier, which may lead to severe neurotoxicity. In addition, occurrence of CRS in patients receiving CAR-T therapy is unpredictable with respect to timing, making it challenging to decide when tocilizumab should be given to the patient. It would be life threatening if a patient develops CRS and does not have immediate access to tocilizumab treatment.

The present disclosure is based, at least in part, on the identification of specific anti-IL-6 or anti-IL-6R antibodies which showed superior effects in inhibiting the IL-6 signaling as relative to other IL-6 antagonistic antibodies. Such anti-IL-6 and anti-IL-6R antibodies would be expected to be more effective in reducing side effects associated with T cell immune therapy mediated by the IL-6 signaling. Without being bound by theory, the methods disclosed herein involve automatic production of IL-6 antagonist (e.g., those disclosed herein) alone with the CAR-T therapy. When CAR-T cells target and kill tumor cells, the CAR-T cells would produce any of the IL-6 antagonists as disclosed herein, for example, a scFv antibody binding to IL-6. Meanwhile, CAR-T cell mediated-killing of tumor cells would stimulate the host immune system to release IL-6. Time-wise, the IL-6 antagonist production by the CAR-T cells would be earlier than IL-6 release by the host immune system, which would be expected to achieve better neutralization effect against IL-6 release. The clinical data provided herein has demonstrated the successful neutralization of IL-6 storm during CAR-T therapy, making tocilizumab treatment unnecessary.

Genetically engineered immune cells (e.g., T cells) co-expressing a CAR targeting a cancer antigen (e.g., CD19 or BCMA), the IL-6 antagonist, and optionally an IL-1 antagonist, as disclosed herein showed superior therapeutic effects in human patients having various types of cancer, including leukemia, non-Hodgkin's lymphoma, and multiple myeloma. Patients who received the CAR-T therapy showed reduced CRS severity, reduced or no neurotoxicity, and/or reduced or no other side effects commonly associated with CAR-T cell therapy (e.g., fever, hypoxia, and/or hypotension), even in the absence of treatment involving anti-CRS agents such as IL-6 antagonists.

Accordingly, provided herein are modified immune cells expressing a chimeric antigen receptor (CAR) and one or more of the IL-6 antagonistic antibodies as those described herein and therapeutic applications thereof.

### I. Modified Immune Cells

One aspect of the present disclosure provides modified immune cells having reduced inflammatory properties compared to wild-type immune cells of the same type. Wild-type cells refer to those that have no such knock-in and knock-out modifications. Such modified immune cells may comprise knock-in of one or more IL-6 antagonistic antibodies as disclosed herein), and optionally a chimeric antigen receptor (CAR) specific to an antigen of interest (e.g., a cancer antigen). In some instances, the modified immune cells may further comprise knock-in of one or more IL-1 antagonists, e.g., IL-1RA or others known in the art or disclosed herein. In some embodiments, the modified immune cells may further comprise one or more knock-out modifications of endogenous genes (e.g., GM-CSF and/or TCR). In other embodiments, the modified immune cells may comprise a wild-type endogenous GM-CSF gene, a wild type TCR gene, or both.

(i) Antagonistic IL-6 Antibodies

IL-6 signals through a complex comprising the membrane glycoprotein gp130 and the IL-6 receptor (IL6R) (see, e.g., Hibi et al., Cell, 63(6):1149-57, 1990). IL-6 binding to IL6R on target cells promotes gp130 homodimerization and subsequent signal transduction. As used herein, IL6R includes both membrane bound and soluble forms of IL6R (sIL6R). When bound to IL-6, soluble IL6R (sIL6R) acts as an agonist and can also promote gp130 dimerization and signaling. Transsignaling can occur whereby sIL-6R secretion by a particular cell type induces cells that only express gp130 to respond to IL-6 (see, e.g., Taga et al., Annu Rev Immunol., 15:797-819, 1997; and Rose-John et al., Biochem J., 300 (Pt 2):281-90, 1994). In one example, sIL6R comprises the extracellular domain of human IL6R (see e.g., Peters et al., J Exp Med., 183(4):1399-406, 1996).

In some embodiments, the modified immune cells disclosed herein express an antagonist IL-6 antibody, which can be an antibody binding to IL-6 or binding to an IL-6 receptor (IL-6R). Such antibodies (antagonistic antibodies) can interfere with binding of IL-6/IL-6R on immune cells, thereby suppressing cell signaling mediated by IL-6.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs.

An antibody (interchangeably used in plural form) as used herein is an immunoglobulin molecule capable of specific binding to a target protein, e.g., IL-6 or IL-6R, through at least one antigen recognition site, located in the variable region of the immunoglobulin molecule. As used herein, the term “antibody” encompasses not only intact (i.e., full-length) antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, the antibodies described herein may specifically bind a target protein or a receptor thereof. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target cytokine if it binds with greater affinity, avidity, more readily, and/or with greater duration than it binds to other substances. For example, an antibody that specifically (or preferentially) binds to an IL-6 or an IL-6R epitope is an antibody that binds this IL-6 epitope or IL-6R epitope with greater affinity, avidity, more readily, and/or with greater duration than it binds to other IL-6 epitopes, non-IL-6 epitopes, other IL-6R epitopes or non-IL-6R epitopes. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

In some embodiments, an antagonistic antibody of a target protein as described herein has a suitable binding affinity for the target protein (e.g., human IL-6 or human IL-6R) or antigenic epitopes thereof. As used herein, “binding affinity” refers to the apparent association constant or KA. The KA is the reciprocal of the dissociation constant (KD). The antagonistic antibody described herein may have a binding affinity (KD) of at least 10⁻⁵, 10⁻⁶, 10⁻⁷, 10⁻⁸, 10⁻⁹, 10⁻¹⁰ M, or lower for the target antigen or antigenic epitope. An increased binding affinity corresponds to a decreased KD. Higher affinity binding of an antibody for a first antigen relative to a second antigen can be indicated by a higher KA (or a smaller numerical value KD) for binding the first antigen than the KA (or numerical value KD) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the antagonistic antibodies described herein have a higher binding affinity (a higher KA or smaller KD) to the target protein in mature form as compared to the binding affinity to the target protein in precursor form or another protein, e.g., an inflammatory protein in the same family as the target protein. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the IL-6 antagonistic antibody as described herein can bind and inhibit the IL-6 signaling by at least 50% (e.g., 60%, 70%, 80%, 90%, 95% or greater). The inhibitory activity of an IL-6 antagonistic antibody described herein can be determined by routine methods known in the art.

The antibodies described herein can be murine, rat, human, or any other origin (including chimeric or humanized antibodies). Such antibodies are non-naturally occurring, i.e., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six), which are altered with respect to the original antibody, which are also termed one or more CDRs “derived from” one or more CDRs from the original antibody. Humanized antibodies may also involve affinity maturation.

The heavy chain variable domains (V_{H}) and light chain variable domains (V_{L}) of exemplary anti-IL-6 antibodies and anti-IL-6R antibodies are provided below (Reference Antibodies 1-6) with the CDRs shown in boldface (determined following the rules described at bioinf.org.uk/abs/):

Antibody 1 (binding to IL-6R):
V_{H} (SEQ ID NO: 1):
EVQLVESGGGLVQPGRSLRLSCAASRFTFDDYAMHWVRQAPGKGLEWVS
GISWNSGRIGYADSVKGRFTISRDNAENSLFLQMNGLRAEDTALYYCAK
GRDSFDIWGQGTMVTVSS
V_{L} (SEQ ID NO: 2):
DIQMTQSPSSVSASVGDRVTITCRASQGISSWLAWYQQKPGKAPKLLIY
GASSLESGVPSRFSGSGSGTDFTLTISSLQPEDFASYYCQQANSFPYTF
GQGTKLEIK
Antibody 2 (binding to IL-6):
V_{H} (SEQ ID NO: 3):
EVQLVESGGGLVQPGGSLRLSCAASGFTFSPFAMSWVRQAPGKGLEWVA
KISPGGSWTYYSDTVTGRFTISRDNAKNSLYLQMNSLRAEDTAVYYCAR
QLWGYYALDIWGQGTTVTVSS
V_{L} (SEQ ID NO: 4):
EIVLTQSPATLSLSPGERATLSCSASISVSYMYWYQQKPGQAPRLLIY
DMSNLASGIPARFSGSGSGTDFTLTISSLEPEDFAVYYCMQWSGYPYT
FGGGTKVEIK
Antibody 3 (binding to IL-6):
V_{H} (SEQ ID NO: 5):
EVQLVESGGKLLKPGGSLKLSCAASGFTFSSFAMSWFRQSPEKRLEWVA
EISSGGSYTYYPDTVTGRFTISRDNAKNTLYLEMSSLRSEDTAMYYCAR
GLWGYYALDYWGQGTSVTVSS
V_{L} (SEQ ID NO: 6):
QIVLIQSPAIMSASPGEKVTMTCSASSSVSYMYWYQQKPGSSPRLLIY
DTSNLASGVPVRFSGSGSGTSYSLTISRMEAEDAATYYCQQWSGYPYT
FGGGTKLEIK
Antibody 4 (binding to IL-6R):
V_{H} (SEQ ID NO: 7):
QVQLQESGPGLVRPSQTLSLTCTVSGYSITSDHAWSWVRQPPGRGLEWI
GYISYSGITTYNPSLKSRVTMLRDTSKNQFSLRLSSVTAADTAVYYCAR
SLARTTAMDYWGQGSLVTVSS
V_{L} (SEQ ID NO: 8):
DIQMTQSPSSLSASVGDRVTITCRASQDISSYLNWYQQKPGKAPKLLIY
YTSRLHSGVPSRFSGSGSGTDFTFTISSLQPEDIATYYCQQGNTLPYTF
GQGTKVEIK
Antibody 5 (binding to IL-6):
V_{H} (SEQ ID NO: 9):
EVQLVESGGGLVQPGGSLRLSCAASGFSLSNYYVTWVRQAPGKGLEWVG
IIYGSDETAYATSAIGRFTISRDNSKNTLYLQMNSLRAEDTAVYYCAR
DDSSDWDAKFNLWGQGTLVTVSS
V_{L} (SEQ ID NO: 10):
AIQMTQSPSSLSASVGDRVTITCQASQSINNELSWYQQKPGKAPKLLIY
RASTLASGVPSRFSGSGSGTDFTLTISSLQPDDFATYYC
QQGYSLRNIDNAFGGGTKVEIK
Antibody 6 (binding to gp130):
V_{H} (SEQ ID NO: 11):
EVQLVESGGGLVQPGGSLRLSCAASGFNFNDYFMNWVRQAPGKGLEWVA
QMRNKNYQYGTYYAESLEGRFTISRDDSKNSLYLQMNSLKTEDTAVYYC
ARESYYGFTSYWGQGTLVTVSS
V_{L} (SEQ ID NO: 12):
DIQMTQSPSSLSASVGDRVTITCQASQDIGISLSWYQQKPGKAPKLLIY
NANNLADGVPSRFSGSGSGTDFTLTISSLQPEDFATYYCLQHNSAPYTF
GQGTKLEIK

In some embodiments, the IL-6 antagonistic antibodies described herein bind to the same epitope in an IL-6 antigen (e.g., human IL-6) or in an IL-6R (e.g., human IL-6R) as one of the reference antibodies provided herein (e.g., Antibody 1 or Antibody 2) or compete against the reference antibody from binding to the IL-6 or IL-6R antigen. Reference antibodies provided herein include Antibodies 1-6, the structural features and binding activity of each of which are provided herein. An antibody that binds the same epitope as a reference antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the reference antibody. Whether two antibodies compete against each other from binding to the cognate antigen can be determined by a competition assay, which is well known in the art. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the IL-6 or IL-6R antigen.

In some instances, the IL-6 antagonistic antibodies disclosed herein may comprise the same heavy chain CDRs and/or the same light chain CDRs as a reference antibody as disclosed herein (e.g., Antibody 1 or Antibody 2). The heavy chain and/or light chain CDRs are the regions/residues that are responsible for antigen-binding; such regions/residues can be identified from amino acid sequences of the heavy chain/light chain sequences of the reference antibody (shown above) by methods known in the art. See, e.g., www.bioinf.org.uk/abs; Almagro, J. Mol. Recognit. 17:132-143 (2004); Chothia et al., J. Mol. Biol. 227:799-817 (1987), as well as others known in the art or disclosed herein. Determination of CDR regions in an antibody is well within the skill of the art, for example, the methods disclosed herein, e.g., the Kabat method (Kabat et al. Sequences of Proteins of Immunological Interest, (5th ed., 1991, National Institutes of Health, Bethesda Md.)) or the Chothia method (Chothia et al., 1989, Nature 342:877; Al-lazikani et al (1997) J. Molec. Biol. 273:927-948)). As used herein, a CDR may refer to the CDR defined by any method known in the art. Two antibodies having the same CDR means that the two antibodies have the same amino acid sequence of that CDR as determined by the same method.

Also within the scope of the present disclosure are functional variants of any of the exemplary anti-IL-6 or anti-IL-6R antibodies as disclosed herein (e.g., Antibody 1 or Antibody 2). A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some examples, the IL-6 antagonistic antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as Antibody 1 or Antibody 2. “Collectively” means that the total number of amino acid variations in all of the three HC CDRs is within the defined range. Alternatively or in addition, the anti-IL-6 or anti-IL6R antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the IL-6 antagonistic antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as Antibody 1 or Antibody 2. In specific examples, the antibody comprises a HC CDR3, which contains no more than 5 amino acid variations (e.g., no more than 4, 3, 2, or 1 amino acid variation) as the HC CDR3 of a reference antibody such as Antibody 1 or Antibody 2. Alternatively or in addition, an IL-6 antagonistic antibody may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: ((a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T4 S; (r) W→Y, F; (s) Y→W, F; and (t) V→I, L.

In some embodiments, the IL-6 antagonistic antibody disclosed herein may comprise heavy chain CDRs that collectively are at least 80% (e.g., 85%, 90%, 95%, or 98%) identical to the heavy chain CDRs of a reference antibody such as Antibody 1 or Antibody 2. Alternatively or in addition, the antibody may comprise light chain CDRs that collectively are at least 80% (e.g., 85%, 90%, 95%, or 98%) identical to the light chain CDRs of the reference antibody. In some embodiments, the IL-6 antagonistic antibody may comprise a heavy chain variable region that is at least 80% (e.g., 85%, 90%, 95%, or 98%) identical to the heavy chain variable region of a reference antibody such as Antibody 1 or Antibody 2 and/or a light chain variable region that is at least 80% (e.g., 85%, 90%, 95%, or 98%) identical to the light chain variable region of the reference antibody.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

The present disclosure also provides germlined variants of any of the reference IL-6 antagonistic antibodies disclosed herein. A germlined variant contains one or more mutations in the framework regions as relative to its parent antibody towards the corresponding germline sequence. To make a germlined variant, the heavy or light chain variable region sequence of the parent antibody or a portion thereof (e.g., a framework sequence) can be used as a query against an antibody germline sequence database (e.g., bioinfo.org.uk/abs/, www.vbase2.org, or imgt.org) to identify the corresponding germline sequence used by the parent antibody and amino acid residue variations in one or more of the framework regions between the germline sequence and the parent antibody. One or more amino acid substitutions can then be introduced into the parent antibody based on the germline sequence to produce a germlined variant.

In some examples, the antagonistic antibodies described herein are human antibodies or humanized antibodies. Alternatively or in addition, the antagonistic antibodies are single-chain antibodies (scFv). Exemplary scFv antibodies are provided below.

IL6/1L6R scFv 1 (SEQ ID NO: 13):
DIQMTQSPSSVSASVGDRVTITCRASQGISSWLAWYQQKPGKAPKLLIYG
ASSLESGVPSRFSGSGSGTDFTLTISSLQPEDFASYYCQQANSFPYTFGQ
GTKLEIKGGGGSGGGGSGGGGSEVQLVESGGGLVQPGRSLRLSCAASRFT
FDDYAMHWVRQAPGKGLEWVSGISWNSGRIGYADSVKGRFTISRDNAENS
LFLQMNGLRAEDTALYYCAKGRDSFDIWGQGTMVTVSS
IL6/1L6R scFv 2 (SEQ ID NO: 14):
EIVLTQSPATLSLSPGERATLSCSASISVSYMYWYQQKPGQAPRLLIYDM
SNLASGIPARFSGSGSGTDFILIISSLEPEDFAVYYCMQWSGYPYTFGGG
TKVEIKGGGGSGGGGSGGGGSEVQLVESGGGLVQPGGSLRLSCAASGFTF
SPFAMSWVRQAPGKGLEWVAKISPGGSWTYYSDTVTGRFTISRDNAKNSL
YLQMNSLRAEDTAVYYCARQLWGYYALDIWGQGTTVTVSS
IL6/1L6R scFv 3 (SEQ ID NO: 15):
QIVLIQSPAIMSASPGEKVIMICSASSSVSYMYWYQQKPGSSPRLLIYDT
SNLASGVPVRFSGSGSGTSYSLTISRMEAEDAATYYCQQWSGYPYTFGGG
TKLEIKGGGGSGGGGSGGGGSEVQLVESGGKLLKPGGSLKLSCAASGFTF
SSFAMSWFRQSPEKRLEWVAEISSGGSYTYYPDTVTGRFTISRDNAKNTL
YLEMSSLRSEDTAMYYCARGLWGYYALDYWGQGTSVTVSS
IL6/1L6R scFv 4 (SEQ ID NO: 16):
QVQLQESGPGLVRPSQTLSLTCTVSGYSITSDHAWSWVRQPPGRGLEWIG
YISYSGITTYNPSLKSRVTMLRDTSKNQFSLRLSSVTAADTAVYYCARSL
ARTTAMDYWGQGSLVTVSSGGGGSGGRASGGGGSGGGGSDIQMTQSPSSL
SASVGDRVTITCRASQDISSYLNWYQQKPGKAPKLLIYYTSRLHSGVPSR
FSGSGSGIDFIFTISSLQPEDIATYYCQQGNTLPYTFGQGTKVEIK

(ii) Antibodies Specific to GM-CSF

Granulocyte-macrophage colony-stimulating factor (GM-CSF), also known as colony-stimulating factor 2 (CSF2), is a monomeric glycoprotein secreted by macrophages, T cells, mast cells, natural killer cells, endothelial cells and fibroblasts. GM-CSF functions as a cytokine and stimulates stem cells to produce granulocytes (e.g., neutrophils, eosinophils, and/or basophils) and monocytes. GM-CSF can trigger the immune/inflammatory cascade, by which activation of a small number of macrophages can rapidly lead to an increase in their numbers, which is a crucial process for fighting infection. GM-CSF may also have effects on mature cells of the immune system, for example, inhibiting neutrophil migration and causing an alteration of the receptors expressed on the cells surface.

In some embodiments, the modified immune cells disclosed herein express an antibody specific to GM-CSF, either alone or in combination with any of the other inhibitory agents disclosed herein, for example, the antagonistic IL-6 antibody, which can be an antibody binding to IL-6 or binding to an IL-6 receptor (IL-6R). Such antibodies (antagonistic antibodies) can suppress cell signaling mediated by GM-CSF, thereby downregulating immune responses triggered by GM-CSF.

The anti-GM-CSF antibody described herein can be of any format and/or from any suitable species, for example, intact (i.e., full-length), antigen-binding fragments thereof (such as Fab, Fab′, F(ab′)2, Fv), single chain (scFv), mutants thereof, fusion proteins comprising an antibody portion, humanized antibodies, chimeric antibodies, diabodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. In some embodiments, the anti-GM-CSF antibody described herein may specifically bind a GM-CSF of a particular species, for example, human GM-CSF.

In some embodiments, the anti-GM-CSF antibody described herein may have a suitable binding affinity for the target protein or antigenic epitopes thereof. For example, the anti-GM-CSF antibody may have a binding affinity (KD) of at least 10⁻⁵, 10⁻⁶, 10⁻⁷, 10⁻⁸, 10⁻⁹, 10⁻¹⁰ M, or lower for the target antigen or antigenic epitope (e.g., the human GM-CSF or an antigenic epitope thereof). In some embodiments, the anti-GM-CSF antibody as described herein can bind and inhibit the GM-CSF signaling by at least 50% (e.g., 60%, 70%, 80%, 90%, 95% or greater). The inhibitory activity of an anti-GM-CSF antibody described herein can be determined by routine methods known in the art.

The heavy chain variable domains (V_{H}) and light chain variable domains (V_{L}) of exemplary anti-GM-CSF antibodies are provided below (Reference Antibodies 7-9) with the CDRs shown in boldface (determined following the rules described at bioinf.org.uk/abs/):

Reference Antibody 7:
V_{H} (SEQ ID NO: 17):
QVQLVQSGAEVKKPGASVKVSCKAFGYPFTDYLLHWVRQAPGQGLEWVG
WLNPYSGDTNYAQKFQGRVTMTRDTSISTAYMELSRLRSDDTAVYYCTR
TTLISVYFDYWGQGTMVTVSS
V_{L} (SEQ ID NO: 18):
DIQMTQSPSSVSASVGDRVTIACRASQNIRNILNWYQQRPGKAPQLLIY
AASNLQSGVPSRFSGSGSGTDFTLTINSLQPEDFATYYCQQSYSMPRTF
GGGTKLEIK
Reference Antibody 8:
V_{H} (SEQ ID NO: 19):
EVQLVESGGGLVQPGGSLRLSCAASGFTFSRHWMHWLRQVPGKGPVWVS
RINGAGTSITYADSVRGRFTISRDNANNTLFLQMNSLRADDTALYFCAR
ANSVWFRGLFDYWGQGTPVTVSS
V_{L} (SEQ ID NO: 20):
EIVLTQSPVTLSVSPGERVTLSCRASQSVSTNLAWYQQKLGQGPRLLIY
GASTRATDIPARFSGSGSETEFTLTISSLQSEDFAVYYCQQYDKWPDTF
GQGTKLEIK
Reference Antibody 9:
V_{H} (SEQ ID NO: 21):
QVQLVESGGGLVQPGGSLRLSCAASGFTFSSYWMNWVRQAPGKGLEWVS
GIENKYAGGATYYAASVKGRFTISRDNSKNTLYLQMNSLRAEDTAVYYC
ARGFGTDFWGQGTLVTVSS
V_{L} (SEQ ID NO: 22):
DIELTQPPSVSVAPGQTARISCSGDSIGKKYAYWYQQKPGQAPVLVIY
KKRPSGIPERFSGSNSGNTATLTISGTQAEDEADYYCSAWGDKGMVFG
GGTKLTVLGQ

In some embodiments, the anti-GM-CSF antibodies described herein bind to the same epitope in a GM-CSF antigen as one of the reference antibodies provided herein (e.g., Antibody 9) or compete against the reference antibody from binding to the GM-CSF antigen. Reference antibodies provided herein include Antibodies 7-9, the structural features and binding activity of each of which are provided herein. Such antibodies can be identified as known to those skilled in the art, e.g., those having substantially similar structural features (e.g., complementary determining regions), and/or those identified by assays known in the art. For example, competition assays can be performed using one of the reference antibodies to determine whether a candidate antibody binds to the same epitope as the reference antibody or competes against its binding to the GM-CSF antigen.

In some instances, the anti-GM-CSF antibodies disclosed herein may comprise the same heavy chain CDRs and/or the same light chain CDRs as a reference antibody as disclosed herein (e.g., Antibody 9). The heavy chain and/or light chain CDRs are the regions/residues that are responsible for antigen-binding; such regions/residues can be identified from amino acid sequences of the heavy chain/light chain sequences of the reference antibody (shown above) by methods known in the art. See also above descriptions.

Also within the scope of the present disclosure are functional variants of any of the exemplary anti-GM-CSF antibodies as disclosed herein (e.g., Antibody 9). A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, or a combination thereof) as the reference antibody.

In some examples, the anti-GM-CSF antibody disclosed herein comprises a HC CDR1, a HC CDR2, and a HC CDR3, which collectively contains no more than 10 amino acid variations (e.g., no more than 9, 8, 7, 6, 5, 4, 3, 2, or 1 amino acid variation) as compared with the HC CDR1, HC CDR2, and HC CDR3 of a reference antibody such as Antibody 9. Alternatively or in addition, the anti-GM-CSF antibody may comprise a LC CDR1, a LC CDR2, and a LC CDR3, which collectively contains no more than 10 amino acid variations (e.g., no more than 9, 8, 7, 6, 5, 4, 3, 2 or 1 amino acid variation) as compared with the LC CDR1, LC CDR2, and LC CDR3 of the reference antibody.

In some examples, the anti-GM-CSF antibody disclosed herein may comprise a HC CDR1, a HC CDR2, and a HC CDR3, at least one of which contains no more than 5 amino acid variations (e.g., no more than 4, 3, 2, or 1 amino acid variation) as the counterpart HC CDR of a reference antibody such as Antibody 9. In specific examples, the antibody comprises a HC CDR3, which contains no more than 5 amino acid variations (e.g., no more than 4, 3, 2, or 1 amino acid variation) as the HC CDR3 of a reference antibody such as Antibody 9. Alternatively or in addition, an anti-GM-CSF antibody described herein may comprise a LC CDR1, a LC CDR2, and a LC CDR3, at least one of which contains no more than 5 amino acid variations (e.g., no more than 4, 3, 2, or 1 amino acid variation) as the counterpart LC CDR of the reference antibody. In specific examples, the antibody comprises a LC CDR3, which contains no more than 5 amino acid variations (e.g., no more than 4, 3, 2, or 1 amino acid variation) as the LC CDR3 of the reference antibody. In some instances, the amino acid residue variations can be conservative amino acid residue substitutions.

In some embodiments, the anti-GM-CSF antibody disclosed herein may comprise heavy chain CDRs that collectively are at least 80% (e.g., 85%, 90%, 95%, or 98%) identical to the heavy chain CDRs of a reference antibody such as Antibody 9. Alternatively or in addition, the antibody may comprise light chain CDRs that collectively are at least 80% (e.g., 85%, 90%, 95%, or 98%) identical to the light chain CDRs of the reference antibody. In some embodiments, the anti-GM-CSF antibody may comprise a heavy chain variable region that is at least 80% (e.g., 85%, 90%, 95%, or 98%) identical to the heavy chain variable region of a reference antibody such as Antibody 9 and/or a light chain variable region that is at least 80% (e.g., 85%, 90%, 95%, or 98%) identical to the light chain variable region of the reference antibody. In some embodiments, the anti-GM-CSF antibody described herein can be a germlined variant of any of the exemplary anti-GM-CSF antibodies described herein, for example, Antibody 9.

In some examples, the anti-GM-CSF antibodies described herein are human antibodies or humanized antibodies. Alternatively or in addition, the antagonistic antibodies are single-chain antibodies (scFv). Exemplary scFv antibodies are provided below.

GM-CSF scFv1 (SEQ ID NO: 23):
QVQLVQSGAEVKKPGASVKVSCKAFGYPFTDYLLHWVRQAPGQGLEWVG
WLNPYSGDTNYAQKFQGRVTMTRDTSISTAYMELSRLRSDDTAVYYCTR
TTLISVYFDYWGQGTMVTVSSGGSGGSGGSGGSGGSDIQMTQSPSSVSA
SVGDRVTIACRASQNIRNILNWYQQRPGKAPQLLIYAASNLQSGVPSRF
SGSGSGTDFTLTINSLQPEDFATYYCQQSYSMPRTFGGGTKLEIK
GM-CSF scFv2 (SEQ ID NO: 24):
EVQLVESGGGLVQPGGSLRLSCAASGFTFSRHWMHWLRQVPGKGPVWVS
RINGAGTSITYADSVRGRFTISRDNANNTLFLQMNSLRADDTALYFCAR
ANSVWFRGLFDYWGQGTPVTVSSGGSGGSGGSGGSGGSEIVLTQSPVTL
SVSPGERVTLSCRASQSVSTNLAWYQQKLGQGPRLLIYGASTRATDIPA
RFSGSGSETEFTLTISSLQSEDFAVYYCQQYDKWPDTFGQGTKLEIK
GM-CSF scFv3 (SEQ ID NO: 25):
QVQLVESGGGLVQPGGSLRLSCAASGFTFSSYWMNWVRQAPGKGLEWVS
GIENKYAGGATYYAASVKGRFTISRDNSKNTLYLQMNSLRAEDTAVYYC
ARGFGTDFWGQGTLVTVSSGGSGGSGGSGGSGGSDIELTQPPSVSVAPG
QTARISCSGDSIGKKYAYWYQQKPGQAPVLVIYKKRPSGIPERFSGSNS
GNTATLTISGTQAEDEADYYCSAWGDKGMVFGGGTKLTVLGQ

(iii) Bi-Specific Antibody Specific to IL-6 and GM-CSF

Also provided herein are bi-specific antibodies to both IL-6 and GM-CSF. A bi-specific antibody comprises two binding moieties, one specific to IL-6/IL-6R and the other specific to GM-CSF. The bi-specific antibody can be of any format as known in the art or disclosed herein. In some embodiments, the binding moiety specific to IL-6/IL-6R can be derived from any of the exemplary IL-6 antagonist antibodies described herein (e.g., Antibody 1 or Antibody 2) or a functional variant thereof as also described herein. Alternatively or in addition, the binding moiety specific to GM-CSF can be derived from any of the exemplary anti-GM-CSF antibodies described herein (e.g., Antibody 9) or a functional variant thereof as also described herein.

In some embodiments, the bi-specific antibody described herein can be configured as a single fusion polypeptide, which comprises a first scFv fragment specific to IL-6/IL-6R and a second scFv fragment specific to GM-CSF. The two scFv fragments can be linked via a peptide linker. Exemplary bi-specific antibodies are provided below:

Bi-Specific Ab1 (peptide linker in boldface and
italicized)(SEQ ID NO: 26):
EIVLTQSPATLSLSPGERATLSCSASISVSYMYWYQQKPGQAPRLLIYDM
SNLASGIPARFSGSGSGTDFTLTISSLEPEDFAVYYCMQWSGYPYTFGGG
TKVEIKGGGGSGGGGSGGGGSEVQLVESGGGLVQPGGSLRLSCAASGFTF
SPFAMSWVRQAPGKGLEWVAKISPGGSWTYYSDTVTGRFTISRDNAKNSL
YLQMNSLRAEDTAVYYCARQLWGYYALDIWGQGTTVTVSS [symbol] QVQL
VQSGAEVKKPGASVKVSCKAFGYPFTDYLLHWVRQAPGQGLEWVGWLNPY
SGDTNYAQKFQGRVTMTRDTSISTAYMELSRLRSDDTAVYYCTRTTLISV
YFDYWGQGTMVTVSSGGSGGSGGSGGSGGSDIQMTQSPSSVSASVGDRVT
IACRASQNIRNILNWYQQRPGKAPQLLIYAASNLQSGVPSRFSGSGSGTD
FTLTINSLQPEDFATYYCQQSYSMPRTFGGGTKLEIK
Bi-Specific Ab2 (peptide linker in boldface and
italicized) (SEQ ID NO: 27):
EIVLTQSPATLSLSPGERATLSCSASISVSYMYWYQQKPGQAPRLLIYDM
SNLASGIPARFSGSGSGTDFTLTISSLEPEDFAVYYCMQWSGYPYTFGGG
TKVEIKGGGGSGGGGSGGGGSEVQLVESGGGLVQPGGSLRLSCAASGFTF
SPFAMSWVRQAPGKGLEWVAKISPGGSWTYYSDTVTGRFTISRDNAKNSL
YLQMNSLRAEDTAVYYCARQLWGYYALDIWGQGTTVTVSS [symbol] EVQL
VESGGGLVQPGGSLRLSCAASGFTFSRHWMHWLRQVPGKGPVWVSRINGA
GTSITYADSVRGRFTISRDNANNTLFLQMNSLRADDTALYFCARANSVWF
RGLFDYWGQGTPVTVSSGGSGGSGGSGGSGGSEIVLTQSPVTLSVSPGER
VTLSCRASQSVSTNLAWYQQKLGQGPRLLIYGASTRATDIPARFSGSGSE
TEFTLTISSLQSEDFAVYYCQQYDKWPDTFGQGTKLEIK
Bi-Specific Ab3 (peptide linker in boldface and
italicized)(SEQ ID NO: 28):
EIVLTQSPATLSLSPGERATLSCSASISVSYMYWYQQKPGQAPRLLIYDM
SNLASGIPARFSGSGSGTDFTLTISSLEPEDFAVYYCMQWSGYPYTFGGG
TKVEIKGGGGSGGGGSGGGGSEVQLVESGGGLVQPGGSLRLSCAASGFTF
SPFAMSWVRQAPGKGLEWVAKISPGGSWTYYSDTVTGRFTISRDNAKNSL
YLQMNSLRAEDTAVYYCARQLWGYYALDIWGQGTTVTVSS [symbol] QVQL
VESGGGLVQPGGSLRLSCAASGFTFSSYWMNWVRQAPGKGLEWVSGIENK
YAGGATYYAASVKGRFTISRDNSKNTLYLQMNSLRAEDTAVYYCARGFGT
DFWGQGTLVTVSSGGSGGSGGSGGSGGSDIELTQPPSVSVAPGQTARISC
SGDSIGKKYAYWYQQKPGQAPVLVIYKKRPSGIPERFSGSNSGNTATLTI
SGTQAEDEADYYCSAWGDKGMVFGGGTKLTVLGQ

(iv) IL-1 Antagonist

Interleukin-1 is a cytokine known in the art and includes two isoforms, IL-1α and IL-1β. IL-1 plays important roles in up- and down-regulation of acute inflammation, as well as other biological pathways.

In some embodiments, the IL-1 antagonist expressed in the modified immune cells disclosed herein can be an interleukin-1 receptor antagonist (IL-1RA). IL-1RA is a naturally-occurring polypeptide, which can be secreted by various types of cells, such as immune cells, epithelial cells, and adipocytes. It binds to cell surface IL-1R receptor and thereby preventing the cell signaling triggered by IL-1/IL-1R interaction. A human IL-1RA is encoded by the IL1RN gene. Below is an exemplary amino acid sequence of a human IL-1RA (SEQ ID NO:29):

[symbol]

RPSGRKSSKMQAFRIWDVNQKTFYLRNNQLVAGYLQGPNVNLE
EKIDVVPIEPHALFLGIHGGKMCLSCVKSGDETRLQLEAVNITDLSENRK
QDKRFAFIRSDSGPTTSFESAACPGWELCTAMEADQPVSLTNMPDEGVMV
IKEYFQEDE

The N-terminal fragment in boldface and italicized refers to the signal peptide in the native IL-1RA. The IL-1RA for use in the instant application may comprise the amino acid sequence corresponding to the mature polypeptide of the human IL-1RA noted above (excluding the signal peptide). Below is an exemplary amino acid sequence of a mature human IL-1RA (SEQ ID NO: 58):

RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE

In some instances, this signal peptide can be replaced with a different signal sequence, for example, MATGSRTSLLLAFGLLCLPWLQEGSA (SEQ ID NO:59). The resultant IL-1RA would have the whole sequence (SEQ ID NO:60):

[symbol]

[symbol]

RPSGRKSSKMQAFRIWDVNQKTFY
LRNNQLVAGYLQGPNVNLEEKIDVVPIEPHALFLGIHGGKMCLSCVKSGD
ETRLQLEAVNITDLSENRKQDKRFAFIRSDSGPTTSFESAACPGWFLCTA
MEADQPVSLTNMPDEGVMVTKFYFQEDE

Other IL-1 antagonists include, but are not limited to, anti-IL-1α or anti-IL-1β antibodies.

(v) Chimeric Antigen Receptor (CAR)

The modified immune cells disclosed herein may further express a chimeric antigen receptor, which is an artificial cell-surface receptors that redirect binding specificity of immune cells (e.g., T cells) to a pathologic antigen to which the CAR binds, thereby eliminating the target disease cells via, e.g., the effector activity of the immune cells. A CAR construct often comprises an extracellular antigen binding domain fused to at least an intracellular signaling domain Cartellieri et al., J Biomed Biotechnol 2010:956304, 2010. The extracellular antigen binding domain, which can be a single-chain antibody fragment (scFv), is specific to an antigen of interest (e.g., a pathologic antibody such as a cancer antigen) and the intracellular signaling domain can mediate a cell signaling that lead to activation of immune cells. As such, immune cells expressing a CAR construct specific to an antigen of interest can bind to diseased cells (e.g., tumor cells) expressing the antigen, leading to activation of the immune cells and elimination of the diseased cells. In some embodiments, the extracellular antigen binding domain targets a tumor antigen, such as CD19 or BCMA. In specific examples, the extracellular antigen binding domain is a single-chain antibody fragment binding to CD19, for example, SEQ ID NO:52. In other examples, the extracellular antigen binding domain is a single-chain antibody fragment binding to BCMA, for example, SEQ ID NO:57.

The CAR construct disclosed herein may comprise one or more intracellular signaling domains. In some examples, CAR comprises an intracellular signaling domain that includes an immunoreceptor tyrosine-based activation motif (ITAM). Such an intracellular signaling domain may be from CD3ζ, CD3δ/ε, CD3γ/ε, or the intracellular signaling domain from a MHC class I molecule or a suitable receptor, for example, a TNF receptor, an immunoglobulin-like receptor, an Fc receptor, a cytokine receptor, an activating NK cell receptor, BTLA, an integrin, or a toll-ligand receptor. In addition, the CAR construct may further comprise one or more co-stimulatory signaling domains, which may be from a co-stimulatory receptor, for example, a signaling lymphocytic activation molecule (SLAM), OX40, CD2, CD7, CD27, CD28, CD30, CD40, CDS, ICAM-1, LFA-1 (CDlla/CD18), 4-1BB (CD137), B7-H3, CDS, ICAM-1, ICOS(CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1 Id, ITGAE, CD103, ITGAL, CDlla, LFA-1, ITGAM, CDllb, ITGAX, CDllc, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, or a ligand that specifically binds with CD83.

The CAR construct disclosed herein may further comprise a transmembrane-hinge domain, which can be obtained from a suitable cell-surface receptor, for example, CD28 or CD8. In some instances, the CAR construct may further comprise a hinge domain, which can be located between the transmembrane domain and the intracellular signaling domain.

(vi) Immune Cells with Knock-in Modifications of IL-6 Antagonistic Antibodies, Anti-GM-CSF Antibodies, and/or IL-1 Antagonists

Also provided herein is a population of immune cells which comprises modified cells having knock-in modifications of an IL-6 antagonistic antibody (e.g., scFv1 or scFv2), an anti-GM-CSF antibody, an IL-1 antagonist, or a combination thereof as described herein, and optionally a CAR construct. In some instances the IL-6 antagonistic antibody and the anti-GM-CSF antibody can be in the form of a bi-specific antibody, e.g., those described herein.

In some embodiments, the genetically modified immune cells may comprise knock-in modifications of an IL-6 antagonist (e.g., an anti-IL-6 antagonistic antibody such as scFv1 or scFv2) and an IL-1 antagonist such as IL-1RA. Such immune cells may have an endogenous GM-CSF gene and optionally also an endogenous TCR gene. Alternatively, the immune cells may have an endogenous GM-CSF gene and a disrupted endogenous TCR gene.

The modified immune cells disclosed herein comprise knock-in modifications to express an antagonistic IL-6 antibody, an anti-GM-CSF antibody, a bi-specific antibody binding to IL-6/IL-6R and GM-CSF, an IL-1 antagonist as disclosed herein, or a combination thereof. Knock-in modifications may comprise delivering to host cells (e.g., immune cells as described herein) one or more exogenous nucleic acids coding for the IL-6 antagonist antibodies, the anti-GM-CSF antibody, the bi-specific antibody, the IL-1 antagonist as disclosed herein, or a combination thereof. The exogenous nucleic acids are in operative linkage to suitable promoters such that the encoded proteins (e.g., cytokine antagonists and/or immune suppressive cytokines) can be expressed in the host cells. In some instances, the exogenous nucleic acids coding for the IL-6 antagonistic antibodies may integrate into the genome of the host cells. In other instances, the exogenous nucleic acids may remain extrachromosomal (not integrated into the genome).

In some instances, any of the modified immune cells may comprise a further knock-in modification to express a CAR construct as disclosed herein.

The modified immune cells comprising one or more knock-in modifications may comprise one or more exogenous nucleic acids (e.g., exogenous expression cassettes) for expressing immune suppressive cytokines and/or antagonists of one or more target inflammatory proteins as described herein. For purpose of the present disclosure, it will be explicitly understood that the term “antagonist” encompass all the previously identified terms, titles, and functional states and characteristics whereby the target protein itself, a biological activity of the target protein, or the consequences of the biological activity, are substantially nullified, decreased, or neutralized in any meaningful degree, e.g., by at least 20%, 50%, 70%, 85%, 90%, or above.

The modified immune cells disclosed herein may further comprise knock-out of one or more inflammatory proteins (e.g., inflammatory cytokines or soluble receptors thereof, inflammatory growth factors, or cytotoxic molecules), knock-in of one or more antagonists of the inflammatory proteins or immune suppressive cytokines, or a combination thereof.

Exemplary inflammatory cytokines or a soluble receptor thereof include interleukin 1 alpha (IL1α), interleukin 1 beta (IL1β), interleukin 2 (IL-2), interleukin 5 (IL-5), interleukin 6 (IL-6), interleukin 7 (IL-7), interleukin 8 (IL-8), interleukin 9 (IL-9), interleukin (IL-12), interleukin 15 (IL-15), interleukin 17 (IL-17), interleukin 18 (IL-18), interleukin 21 (IL-21), interleukin 23 (IL-23), sIL-1RI, sIL-2Rα, soluble IL-6 receptor (sIL6R), interferon α (IFNα), interferon β (IFNβ), interferon γ (IFNγ), Macrophage inflammatory proteins (e.g., MIPα and MIPβ), Macrophage colony-stimulating factor 1 (CSF1), leukemia inhibitory factor (LIF), granulocyte colony-stimulating factor (G-CSF), granulocyte-macrophage colony-stimulating factor (GM-CSF), C-X-C motif chemokine ligand 10 (CXCL10), chemokine (C-C motif) ligand 5 (CCL5), eotaxin, tumor necrosis factor (TNF), monocyte chemoattractant protein 1 (MCP1), monokine induced by gamma interferon (MIG), receptor for advanced glycation end-products (RAGE), c-reactive protein (CRP), angiopoietin-2, and von Willebrand factor (VWF).

Examples of target inflammatory proteins include, but are not limited to, inflammatory cytokines or soluble receptors thereof (e.g., IL2, IL1α, IL1β, IL-5, IL-6, IL-7, IL-8, IL-9, IL-12, IL-15, IL-17, IL-18, IL-21, IL-23, sIL-1RI, sIL-2Rα, sIL6R, IFNα, IFNβ, IFNγ, MIPα, MIPβ, CSF1, LIF, G-CSF, GM-CSF, CXCL10, CCL5, eotaxin, TNF, MCP1, MIG, RAGE, CRP, angiopoietin-2, and VWF), inflammatory growth factors (e.g., TGFα, VEGF, EGF, HGF, and FGF) and cytotoxic molecules (e.g., perforin, granzyme, and ferritin).

The immune cell population as described herein can be further modified to express an exogenous cytokine, a chimeric synNotch receptor, a chimeric immunoreceptor, a chimeric costimulatory receptor, a chimeric killer-cell immunoglobulin-like receptor (KIR), and/or an exogenous T cell receptor. This can be done either before, after, or concurrently with the knock-in and/or knock-out modifications. Such receptors may be cloned and integrated into any suitable expression vector using routine recombinant technology. Considerations for design of chimeric antigen receptors are also known in the art. See, e.g., Sadelain et al., Cancer Discov., 3(4):388-98, 2013.

The immune cells disclosed herein can be T-cells, NK cells, dendritic cells, macrophages, B cells, neutrophils, eosinophils, basophils, mast cells, myeloid-derived suppressor cells, mesenchymal stem cells, precursors thereof, or combinations thereof.

### II. Methods of Preparing Modified Immune Cells

Any of the knock-in and knock-out modifications may be introduced into suitable immune cells by routine methods and/or approaches described herein. Typically, such methods would involve delivery of genetic material into the suitable immune cells to either down-regulate expression of a target endogenous inflammatory protein, express a cytokine antagonist of interest or express an immune suppressive cytokine of interest.

(A) Knocking In Modification

To generate a knock-in of one or more cytokine antagonists described herein, a coding sequence of any of the antagonists and/or immune suppressive cytokines described herein may be cloned into a suitable expression vector (e.g., including but not limited to lentiviral vectors, retroviral vectors, adenovivral vectors, adeno-associated vectors, PiggyBac transposon vector and SleepingBeauty transposon vector) and introduced into host immune cells using conventional recombinant technology. Sambrook et al., Molecular Cloning, A Laboratory Manual, 3rd Ed., Cold Spring Harbor Laboratory Press. As a result, modified immune cells of the present disclosure may comprise one or more exogenous nucleic acids encoding at least one cytokine antagonist or at least one immune suppressive cytokine. In some instances, the coding sequence of one or more antagonists and/or one or more immune suppressive cytokines is integrated into the genome of the cell. In some instances, the coding sequence of one or more antagonists is not integrated into the genome of the cell.

An exogenous nucleic acid comprising a coding sequence of a cytokine antagonist or an immune suppressive cytokine of interest may further comprise a suitable promoter, which can be in operable linkage to the coding sequence. A promoter, as used herein, refers to a nucleotide sequence (site) on a nucleic acid to which RNA polymerase can bind to initiate the transcription of the coding DNA (e.g., for a cytokine antagonist) into mRNA, which will then be translated into the corresponding protein (i.e., expression of a gene). A promoter is considered to be “operably linked” to a coding sequence when it is in a correct functional location and orientation relative to the coding sequence to control (“drive”) transcriptional initiation and expression of that coding sequence (to produce the corresponding protein molecules). In some instances, the promoter described herein can be constitutive, which initiates transcription independent other regulatory factors. In some instances, the promoter described herein can be inducible, which is dependent on regulatory factors for transcription. Exemplary promoters include, but are not limited to ubiquitin, RSV, CMV, EF1α and PGK1. In one example, one or more nucleic acids encoding one or more antagonists of one or more inflammatory cytokines as those described herein, operably linked to one or more suitable promoters can be introduced into immune cells via conventional methods to drive expression of one or more antagonists.

Additionally, the exogenous nucleic acids described herein may further contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Suitable methods for producing vectors containing transgenes are well known and available in the art. Sambrook et al., Molecular Cloning, A Laboratory Manual, 3rd Ed., Cold Spring Harbor Laboratory Press.

In some instances, multiple cytokine antagonists as described herein can be constructed in one expression cassette in a multicistronic manner such that the multiple cytokine antagonists as separate polypeptides. In some examples, an internal ribosome entry site can be inserted between two coding sequences to achieve this goal. Alternatively, a nucleotide sequence coding for a self-cleaving peptide (e.g., T2A or P2A) can be inserted between two coding sequences. Exemplary designs of such multicistronic expression cassettes are provided in Examples below.

(B) Knocking Out Modification

Any methods known in the art for down-regulating the expression of an endogenous gene in a host cell can be used to reduce the production level of a target endogenous cytokine/protein as described herein. A gene editing method may involve use of an endonuclease that is capable of cleaving the target region in the endogenous allele. Non-homologous end joining in the absence of a template nucleic acid may repair double-strand breaks in the genome and introduce mutations (e.g., insertions, deletions and/or frameshifts) into a target site. Gene editing methods are generally classified based on the type of endonuclease that is involved in generating double stranded breaks in the target nucleic acid. Examples include, but are not limited to, Clustered Regularly Interspaced Short Palindromic Repeats (CRISPR)/endonuclease systems, transcription activator-like effector-based nuclease (TALEN), zinc finger nucleases (ZFN), endonucleases (e.g., ARC homing endonucleases), meganucleases (e.g., mega-TALs), or a combination thereof.

Various gene editing systems using meganucleases, including modified meganucleases, have been described in the art; see, e.g., the reviews by Steentoft et al., Glycobiology 24(8):663-80, 2014; Belfort and Bonocora, Methods Mol Biol. 1123:1-26, 2014; Hafez and Hausner, Genome 55(8):553-69, 2012; and references cited therein. In some examples, a knocking-out event can be coupled with a knocking-in event—an exogenous nucleic acid coding for a desired molecule such as those described herein can be inserted into a locus of a target endogenous gene of interest via gene editing.

In some instances, knocking-out an endogenous gene can be achieved using the CRISPR technology. Exemplary target endogenous genes include IL-2, GM-CSF, TNFA T-cell receptor, β2M, etc. Exemplary gRNAs for use in knocking-out target endogenous genes IL-2, GM-CSF, and TNFA are provided in Examples below.

Alternatively, any of the knock-out modification may be achieved using antisense oligonucleotides (e.g., interfering RNAs such as shRNA or siRNA) or ribozymes via methods known in the art. An antisense oligonucleotide specific to a target cytokine/protein refers to an oligonucleotide that is complementary or partially complementary to a target region of an endogenous gene of the cytokine or an mRNA encoding such. Such antisense oligonucleotides can be delivered into target cells via conventional methods. Alternatively, expression vectors such as lentiviral vectors or equivalent thereof can be used to express such an antisense oligonucleotides.

(C) Preparation of Immune Cell Population Comprising Modified Immune Cells

A population of immune cells comprising any of the modified immune cells described herein, or a combination thereof, may be prepared by introducing into a population of host immune cells one or more of the knock-in modifications, one or more of the knock-out modifications, or a combination thereof. The knock-in and knock-out modifications can be introduced into the host cells in any order.

In some instances, one or more modifications are introduced into the host cells in a sequential manner without isolation and/or enrichment of modified cells after a preceding modification event and prior to the next modification event. In that case, the resultant immune cell population may be heterogeneous, comprising cells harboring different modifications or different combination of modifications. Such an immune cell population may also comprise unmodified immune cells. The level of each modification event occurring in the immune cell population can be controlled by the amount of genetic materials that induce such modification as relative to the total number of the host immune cells. See also above discussions.

In other instances, modified immune cells may be isolated and enriched after a first modification event before performing a second modification event. This approach would result in the production of a substantially homogenous immune cell population harboring all of the knock-in and/or knock-out modifications introduced into the cells.

In some examples, the knock-in modification(s) and the knock-out modification(s) are introduced into host immune cells separately. For example, a knock-out modification is performed via gene editing to knock out an endogenous gene for a target cytokine and a knock-in modification is performed by delivering into the host immune cells a separate exogenous expression cassette for producing one or more cytokine antagonists. In some instances, the knock-in and knock-out event can be occurred simultaneously, for example, the knock-in cassette can be inserted into the locus of a target gene to be knocked-out.

### III. Therapeutic Applications

Any of the immune cell populations comprising the modified immune cells as described herein may be used in an adoptive immune cell therapy for treating a target disease, such as leukemia or lymphoma. Due to the knock-in and knock-out modifications introduced in to the immune cells, particularly the knock-in of the IL-6 antagonistic antibody, the anti-GM-CSF antibody (maybe a fragment of a bi-specific antibody, which also contains a fragment binding to IL-6 or IL-6R), the IL-1 antagonist as described herein, or a combination thereof, the therapeutic uses of such would be expected to reduce cytotoxicity associated with conventional adoptive immune cell therapy (reducing inflammatory cytokines produced by both the immune cells used in adoptive immune cell therapy and endogenous immune cells of the recipient, which can be activated by the infused immune cells), while achieving the same or better therapeutic effects.

To practice the therapeutic methods described herein, an effective amount of the immune cell population, comprising any of the modified immune cells as described herein, may be administered to a subject who needs treatment via a suitable route (e.g., intravenous infusion). The immune cell population may be mixed with a pharmaceutically acceptable carrier to form a pharmaceutical composition prior to administration, which is also within the scope of the present disclosure. The immune cells may be autologous to the subject, i.e., the immune cells are obtained from the subject in need of the treatment, modified to reduce expression of one or more target cytokines/proteins, for example, those described herein, to express one or more cytokine antagonists described herein, to express a CAR construct and/or exogenous TCR, or a combination thereof. The resultant modified immune cells can then be administered to the same subject. Administration of autologous cells to a subject may result in reduced rejection of the immune cells as compared to administration of non-autologous cells. Alternatively, the immune cells can be allogeneic cells, i.e., the cells are obtained from a first subject, modified as described herein and administered to a second subject that is different from the first subject but of the same species. For example, allogeneic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep or monkey). The subject may be suffering from cancer, have an infectious disease or an immune disorder. Exemplary cancers include but are not limited to hematologic malignancies (e.g., B-cell acute lymphoblastic leukemia, chronic lymphocytic leukemia and multiple myeloma). Exemplary infectious diseases include but are not to human immunodeficiency virus (HIV) infection, Epstein-Barr virus (EBV) infection, human papillomavirus (HPV) infection, dengue virus infection, malaria, sepsis and E. coli infection. Exemplary immune disorders include but are not limited to, autoimmune diseases, such as rheumatoid arthritis, type I diabetes, systemic lupus erythematosus, inflammatory bowel disease, multiple sclerosis, Guillain-Barre syndrome, chronic inflammatory demyelinating polyneuropathy, psoriasis, Graves' disease, Hashimoto's thyroiditis, myasthenia gravis, and vasculitis.

In some examples, the subject to be treated in the methods disclosed herein may be a human cancer patient. For example, the human patient may have a cancer of B-cell origin. Examples include B-lineage acute lymphoblastic leukemia, B-cell chronic lymphocytic leukemia and B-cell non-Hodgkin's lymphoma. Alternatively, the human patient may have breast cancer, gastric cancer, neuroblastoma, or osteosarcoma.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

In some instances, the genetically engineered immune cells as disclosed herein are for use in treating cancer. Such immune cells express a chimeric antigen receptor (CAR) that targets a cancer antigen, for example, CD19 or BCMA. In addition to any of the IL-6 antagonists disclosed herein (e.g., scFv comprising SEQ ID NO:13 or SEQ ID NO:14), the genetically engineered immune cells may further express an IL-1 antagonist such as an IL-1RA. The genetically engineered immune cells may have endogenous GM-CSF and/or TCR genes knocked out. Alternatively, the genetically engineered immune cells may carry wild-type endogenous GM-CSF and/or TCR genes.

An effective amount of the genetically engineered immune cells may be administered to a human patient in need of the treatment via a suitable route, e.g., intravenous infusion. In some instances, about 1×10⁶ to about 1×10⁸ CAR+ T cells may be given to a human patient (e.g., a leukemia patient, a lymphoma patient, or a multiple myeloma patient). In some examples, a human patient may receive multiple doses of the genetically engineered immune cells. For example, the patient may receive two doses of the immune cells on two consecutive days. In some instances, the first dose is the same as the second dose. In other instances, the first dose is lower than the second dose, or vice versa.

In any of the treatment methods disclosed herein, which involves the use of the genetically engineered immune cells, the subject may be administered IL-2 concurrently with the cell therapy. More specifically, an effective amount of IL-2 may be given to the subject via a suitable route before, during, or after the cell therapy. In some embodiments, IL-2 is given to the subject after administration of the immune cells.

Alternatively or in addition, the subject being treated by the cell therapy disclosed herein may be free from treatment involving an IL-6 antagonist (aside from the IL-6 antagonist produced by the immune cells used in the cell therapy) after immune cell infusion.

The immune cell populations comprising the modified immune cells as described herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

In some examples, the subject is subject to a suitable anti-cancer therapy (e.g., those disclosed herein) to reduce tumor burden prior to the CAR-T therapy disclosed herein. For example, the subject (e.g., a human cancer patient) may be subject to a chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell therapy disclosed herein may be monitored via routine methods. If a patient showed a high growth rate of cancer cells after the initial anti-cancer therapy and/or after the CAR-T therapy, the patient may be subject to a new round of chemotherapy to reduce tumor burden followed by any of the CAR-T therapy as disclosed herein.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### IV. Kits for Therapeutic Uses or Making Modified Immune Cells

The present disclosure also provides kits for use of any of the target diseases described herein involving the immune cell population described herein and kits for use in making the modified immune cells as described herein.

A kit for therapeutic use as described herein may include one or more containers comprising an immune cell population, which may be formulated to form a pharmaceutical composition. The immune cell population comprises any of the modified immune cells described herein or a combination thereof. The population of immune cells, such as T lymphocytes, NK cells, and others described herein may further express a CAR construct and/or an exogenous TCR, as described herein.

In some embodiments, the kit can additionally comprise instructions for use of the immune cell population in any of the methods described herein. The included instructions may comprise a description of administration of the immune cell population or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the immune cell population or the pharmaceutical composition comprising such to a subject who is in need of the treatment.

The instructions relating to the use of the immune cell population or the pharmaceutical composition comprising such as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of immune cells (e.g., T lymphocytes or NK cells) that comprise any of the modified immune cells or a combination thereof.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Also provided here are kits for use in making the modified immune cells as described herein. Such a kit may include one or more containers each containing reagents for use in introducing the knock-in and/or knock-out modifications into immune cells. For example, the kit may contain one or more components of a gene editing system for making one or more knock-out modifications as those described herein. Alternatively or in addition, the kit may comprise one or more exogenous nucleic acids for expressing cytokine antagonists as also described herein and reagents for delivering the exogenous nucleic acids into host immune cells. Such a kit may further include instructions for making the desired modifications to host immune cells.

### V. General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames&S. J. Higgins eds. (1985; Transcription and Translation (B. D. Hames&S. J. Higgins, eds. (1984; Animal Cell Culture (R. I. Freshney, ed. (1986; Immobilized Cells and Enzymes (1RL Press, (1986»; and B. Perbal, A practical Guide To Molecular Cloning (1984); F. M. Ausubel et al. (eds.).

The present disclosure is not limited in its application to the details of construction and the arrangements of component set forth in the description herein or illustrated in the drawings. The present disclosure is capable of other embodiments and of being practice or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items. As also used in this specification and the appended claims, the singular forms “a,” “an,” and “the” include plural references unless the context clearly dictates otherwise.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Effects of IL-6 Antagonistic Antibodies Expressed in 293T Cells in Inhibiting IL-6 Signaling

HEK293T cells were transfected with a 3^{rd} generation self-inactivating (SIN) lentiviral transfer vectors encoding single-chain variable fragment (scFv) antibody derived from reference antibodies 1, 2, 3, and 4 disclosed herein, which target IL-6 or IL-6R, by Lipofectamine 2000 (Thermo Scientific). A CD8 leading sequence is located before the anti-IL6 scFv. See descriptions in Example 7 below. The scFv antibodies were fused with an Fc fragment of human IgG1. The supernatants of transfected cells, containing the scFv antibodies expressed by the transfected HEK293T cells, were collected, diluted, and added to HEK-Blue IL-6 reporter cells (Invivogen) in the presence of 2 ng/ml human IL-6. HEK-Blue IL-6 reporter cells were used because they are capable of producing Secreted Embryonic Alkaline Phosphatase (SEAP) upon human IL-6 stimulation. After overnight incubation, the supernatant of HEK-Blue IL-6 cells was collected and incubated with Quant-Blue substrate solution. SEAP production was quantified by measuring optical absorbance of converted substrate Quant Blue (Invivogen) at 650 nm wavelength through a spectrophotometer.

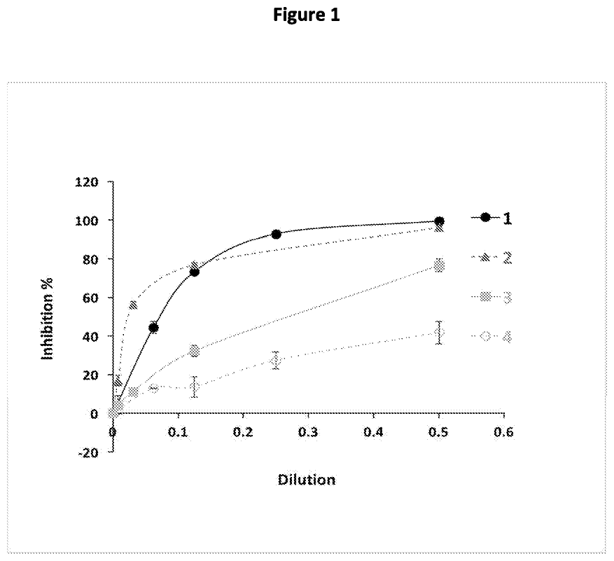

As shown in FIG. 1, all of the scFv antibodies are able to inhibit IL-6 signaling by binding to IL-6 or IL-6R expressed on the reporter cells. Amongst the 4 scFv antibodies tested, the scFv antibodies derived from antibodies 1 and 2 exhibited higher efficiency in inhibiting IL-6 signaling as compared to the scFv antibodies derived from reference antibodies 3 and 4. For example, scFv antibodies derived from reference antibodies 1 and 2 showed close to 100% inhibition of IL-6 signaling at dilution 0.5, whereas those derived from reference antibodies 3 and 4 showed inhibition efficiency lower than 60% at the same dilution. This result suggested that antibody 1 and antibody 2 are more effective in blocking IL-6-IL6R signaling.

### Example 2: Combined Effects of Anti-IL-6 Antibody and IL-1RA Expressed in 293T Cells in Blocking Both IL-6 and IL-1 Signaling

Nucleic acids encoding Construct 1, Construct 2, and Construct 3 were cloned into the 3^{rd} generation self-inactivating (SIN) lentiviral transfer vector Construct 1 includes, from N-terminus to C-terminus, a T2A linker, an scFv antibody derived from reference Antibody 2 (targeting IL-6), a P2A linker, and an IL-1 receptor antagonist (IL-1RA) (T2A-Sir-P2A-IL1RA). Construct 2 contains, from N-terminus to C-terminus, the T2A linker, the scFv antibody, a (G₄S)₃ linker, and the IL1RA (T2A-Sir-(G4S)₃-IL1RA). Construct 3 contains, from N-terminus to C-terminus, the scFv antibody, the (G₄S)₃ linker, the IL1RA, and the T2A linker (Sir-(G₄S)₃-IL1RA-T2A). In construct 1, 2, and 3, a CD8 leading sequence is located before the anti-IL6 scFv. See descriptions in Example 7 below. In construct 1, a hGH leading sequence is located between the P2A and IL1RA.

293T cells were transfected with a lentiviral transfer vector as described above by Lipofectamine 2000 (Thermo Scientific). The supernatant from the transfected cells was collected and added to HEK-Blue IL-1R Cells (Invivogen) in different dilutions as indicated in the presence of 10 pg/ml IL-1B. After overnight incubation, the supernatant of HEK-Blue IL-1R Cells was collected and incubated with substrate solution of Quant-Blue (Invivogen), and the optical absorbance of converted substrate was measured at 650 nm wavelength through a spectrophotometer.

The supernatant was also added to HEK-Blue IL-6 Cells (Invivogen) in different dilutions as indicated in the presence of 2 ng/ml IL-6 After overnight incubation, the supernatant of HEK-Blue IL-6 Cells was collected and incubated with substrate solution of Quant-Blue (Invivogen), and the optical absorbance of converted substrate was measured at 650 nm wavelength through a spectrophotometer.

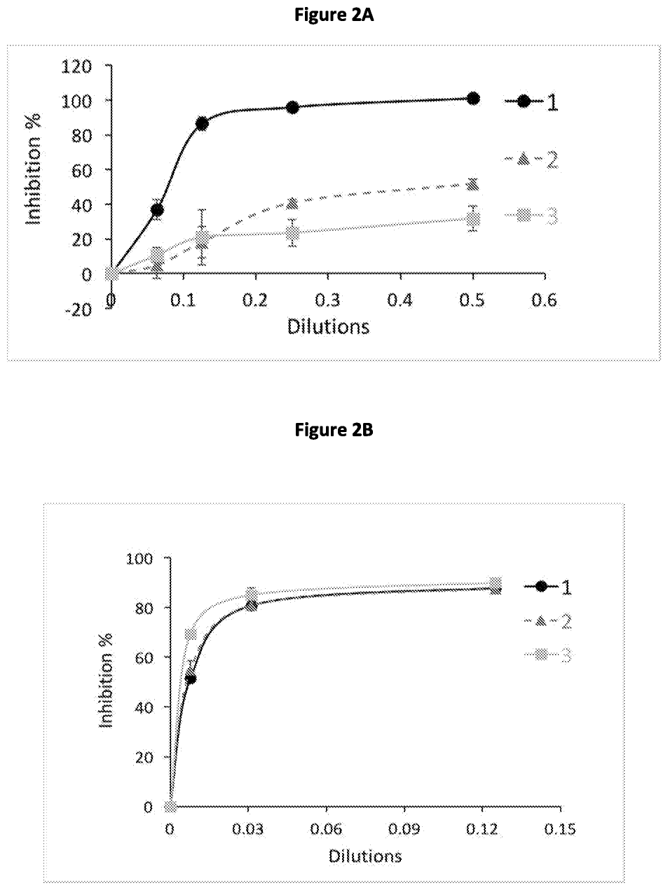

As shown in FIG. 2A and FIG. 2B, the dual-constructs described herein successfully blocked both the IL-1 and IL-6 signaling.

### Example 3: Combined Effects of IL-6 and GM-CSF Antagonistic Antibodies Expressed in 293T Cells in Blocking Both IL-6 and GM-CSF Signaling

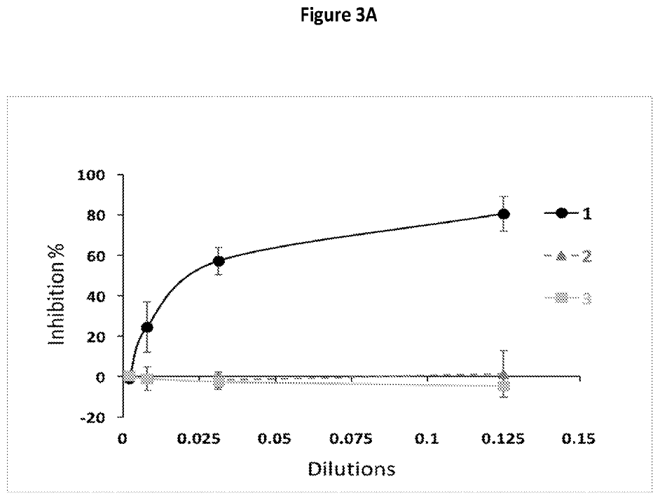

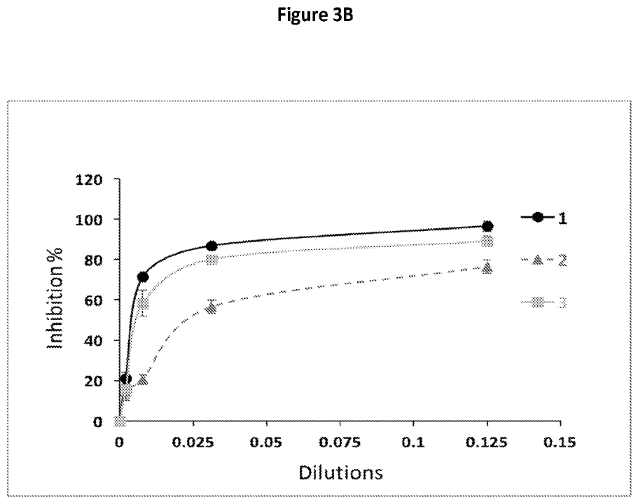

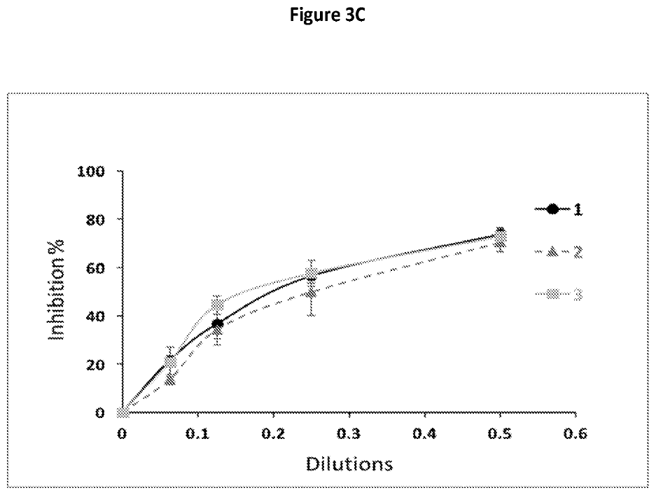

Constructs for expressing three exemplary bispecific antibodies specific to IL-6 and GM-CSF and the IL1RA described in Example 2 above were produced by conventional recombinant technology. Each of the bi-specific antibody contains a scFv derived from reference Antibody 2 (targeting IL-6) and a scFv derived from reference Antibody 7, Antibody 8, or Antibody 9 (all targeting GM-CSF). The two scFv fragments in the bispecific antibody are linked by a GSGGSG linker. Each of the bispecific antibody is linked to the IL1RA via the P2A linker. These constructs are designated at Ab2/Ab9-P2A-IL1RA, Ab2/Ab7-P2A-IL1RA, and Ab2/Ab8-P2A-IL1RA (corresponding to 1, 2, and 3 in FIGS. 3A-3C, respectively), In these constructs, a CD8 leading sequence is located before the anti-IL6 scFv, and a hGH leading sequence is located between the P2A and IL1RA. See descriptions in Example 7 below.

Nucleic acids encoding the above-noted constructs were inserted into the 3^{rd} generation self-inactivating (SIN) lentiviral transfer vector by recombinant technology. The resultant lentiviral transfer vectors were transfected into 293T cells by Lipofectamine 2000 (Thermo Scientific). The supernatant from the transfected cells was collected.

The supernatant was added to TF-1 cells in different dilutions as indicated in the presence of 2 ng/ml GM-CSF for co-culture of 2 days, since TF-1 cells are completely dependent on GM-CSF for proliferation. Then proliferation of TF-1 cells was evaluated by the PromegaCellTiter 96® AQueous One Solution Cell Proliferation Assay.

The supernatant was also added to HEK-Blue IL-6 Cells (Invivogen) in different dilutions as indicated in the presence of 2 ng/ml IL-6. After overnight incubation, the supernatant of HEK-Blue IL-6 Cells was collected and incubated with substrate solution of Quant-Blue (Invivogen), and the optical absorbance of converted substrate was measured at 650 nm wavelength through a spectrophotometer.

Further, the supernatant was added to HEK-Blue IL-1R Cells (Invivogen) in different dilutions as indicated in the presence of 10 pg/ml IL-1B. After overnight incubation, the supernatant of HEK-Blue IL-1R Cells was collected and incubated with substrate solution of Quant-Blue (Invivogen), and the optical absorbance of converted substrate was measured at 650 nm wavelength through a spectrophotometer.

The three constructs tested in this Example all showed inhibitory activity against the IL-6 signaling and the IL-1 signaling. FIGS. 3B and 3C. On the other hand, the construct that contains the Ab2/Ab9bispecific antibody showed significant blockade activity against the GM-CSF signaling. FIG. 3A.

### Example 4: Disruption of GM-CSF by CRISPR Technology

Primary T cells from healthy donor (PPA research) were activated by anti-CD3/28 beads (Thermo scientific). Four days later, activated T cells were electroporated with Cas9 protein (Thermo scientific) plus different gRNA targeting the first exon of GM-CSF as shown below, while T cells electroporated with Cas9 protein only were served as a negative control.

Exemplary guild RNA template sequences for targeting human GM-CSF exon 1 (spacer sequence before the PAM motif is shown in boldface):

gRNA1 (SEQ ID NO: 30):
GCTGCAGAGCCTGCTGCTCTGTTTTAGAGCTAGAAATAGCAAGTTAAAAT
AAGGCTAGTCCGTTATCAACTTGAAAAAGTGGCACCGAGTCGGTGCTTTT
gRNA2 (SEQ ID NO: 31):
GGAGCATGTGAATGCCATCCGTTTTAGAGCTAGAAATAGCAAGTTAAAAT
AAGGCTAGTCCGTTATCAACTTGAAAAAGTGGCACCGAGTCGGTGCTTTT
gRNA3 (SEQ ID NO: 32):
GCATGTGAATGCCATCCAGGGTTTTAGAGCTAGAAATAGCAAGTTAAAAT
AAGGCTAGTCCGTTATCAACTTGAAAAAGTGGCACCGAGTCGGTGCTTTT
gRNA4 (SEQ ID NO: 33):
GAGACGCCGGGCCTCCTGGAGTTTTAGAGCTAGAAATAGCAAGTTAAAAT
AAGGCTAGTCCGTTATCAACTTGAAAAAGTGGCACCGAGTCGGTGCTTTT
gRNA5 (SEQ ID NO: 34):
GATGGCATTCACATGCTCCCGTTTTAGAGCTAGAAATAGCAAGTTAAAAT
AAGGCTAGTCCGTTATCAACTTGAAAAAGTGGCACCGAGTCGGTGCTTTT
gRNA6 (SEQ ID NO: 35):
GCTCCCAGGGCTGCGTGCTGGTTTTAGAGCTAGAAATAGCAAGTTAAAAT
AAGGCTAGTCCGTTATCAACTTGAAAAAGTGGCACCGAGTCGGTGCTTTT
gRNA7 (SEQ ID NO: 36):
GCGTGCTGGGGCTGGGCGAGGTTTTAGAGCTAGAAATAGCAAGTTAAAAT
AAGGCTAGTCCGTTATCAACTTGAAAAAGTGGCACCGAGTCGGTGCTTTT
gRNA8 (SEQ ID NO: 37):
GCTGGGGCTGGGCGAGCGGGGTTTTAGAGCTAGAAATAGCAAGTTAAAAT
AAGGCTAGTCCGTTATCAACTTGAAAAAGTGGCACCGAGTCGGTGCTTTT

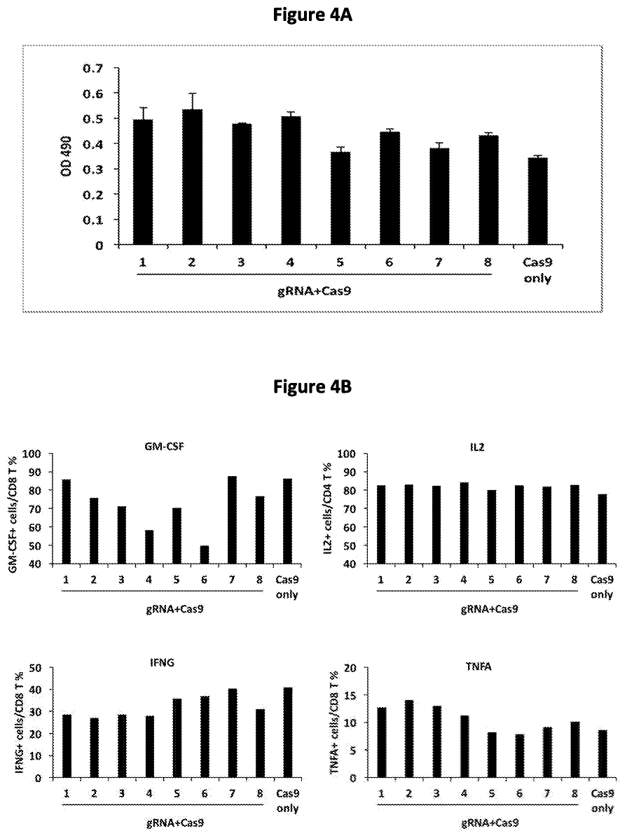

Four days after electroporation, T cells were assessed for proliferation with CellTiter 96® AQueous One Solution Cell Proliferation Assay (MTS) (Promega). The results suggest that gene editing against GM-CSF does not significantly alter T cell proliferation after electroporation in the presence of exogenous IL2. FIG. 4A.

The T cells were also activated with PMA/Ionomycin to analyze expression of cytokines (GM-CSF, IL-2, IFNγ, and TNFα) using intracellular staining kit (Biolegend and BD Bioscience). The results indicate that the GM-CSF gene editing significantly reduced GM-CSF expression but showed no significant impact on IL2, IFNγ or TNFα expression. FIG. 4B.

### Example 5: Disruption of IL-2 by CRISPR Technology

Primary T cells from healthy donor (PPA research) were activated by anti-CD3/28 beads (Thermo scientific). Four days later, activated T cells were electroporated with Cas9 protein (Thermo scientific) plus different gRNA targeting the first exon of IL2 as shown below, while T cells electroporated with Cas9 protein only were served as a negative control.

Exemplary gRNA template sequences for targeting human IL2 exon 1 (spacer sequence before PAM motif shown in boldface):

gRNA1 (SEQ ID NO: 38):
GACTTAGTGCAATGCAAGACGTTTTAGAGCTAGAAATAGCAAGTTAAAAT
AAGGCTAGTCCGTTATCAACTTGAAAAAGTGGCACCGAGTCGGTGCTTTT
gRNA2 (SEQ ID NO: 39):
GATTTACAGATGATTTTGAAGTTTTAGAGCTAGAAATAGCAAGTTAAAAT
AAGGCTAGTCCGTTATCAACTTGAAAAAGTGGCACCGAGTCGGTGCTTTT
gRNA3 (SEQ ID NO: 40):
AAGAAAACACAGCTACAACGTTTTAGAGCTAGAAATAGCAAGTTAAAATA
AGGCTAGTCCGTTATCAACTTGAAAAAGTGGCACCGAGTCGGTGCTTTT
gRNA4 (SEQ ID NO: 41):
CAACTGGAGCATTTACTGCGTTTTAGAGCTAGAAATAGCAAGTTAAAATA
AGGCTAGTCCGTTATCAACTTGAAAAAGTGGCACCGAGTCGGTGCTTTT
gRNA5 (SEQ ID NO: 42):
TCTTTGTAGAACTTGAAGTGTTTTAGAGCTAGAAATAGCAAGTTAAAATA
AGGCTAGTCCGTTATCAACTTGAAAAAGTGGCACCGAGTCGGTGCTTTT

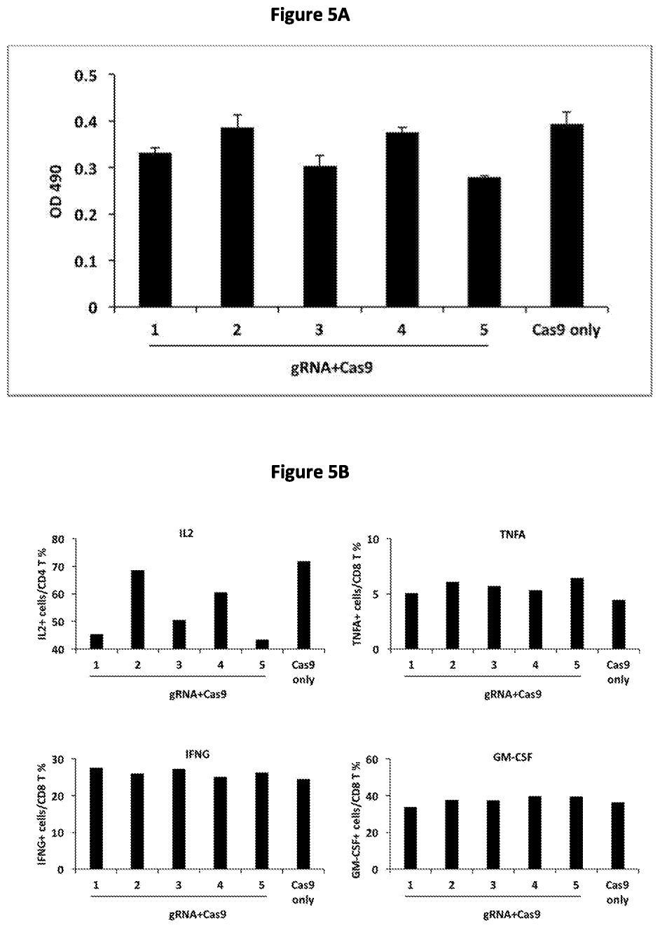

Four days after electroporation, T cells were assessed for proliferation using the CellTiter 96® AQueous One Solution Cell Proliferation Assay (MTS) (Promega). The results show that knocking out IL2 did not significantly alter T cell proliferation after electroporation in the presence of exogenous IL2. FIG. 5A.

The T cells were activated with PMA/Ionomycin to analyze cytokine expression (GM-CSF, IL-2, IFNγ, and TNFα) through intracellular staining kit (Biolegend and BD Bioscience). The results indicate that the IL2 gene editing significantly reduced IL2 expression but showed no significant impact on GM-CSF, IFNγ or TNFA expression. FIG. 5B.

### Example 6: Disruption of TNFα by CRISPR Technology

Primary T cells from healthy donor (PPA research) were activated by anti-CD3/28 beads (Thermo scientific). Three days later, activated T cells are electroporated with Cas9 protein (Thermo scientific) plus different gRNA targeting the first exon of TNFα as shown below, while T cells electroporated with Cas9 only were served as a negative control.

Exemplary gRNA template sequences for targeting human TNFα exon 1 (spacer sequence before PAM motif shown in boldface):

sgRNA 1 (SEQ ID NO: 43):
GAGCACTGAAAGCATGATCCGTTTTAGAGCTAGAAATAGCAAGTTAAAAT
AAGGCTAGTCCGTTATCAACTTGAAAAAGTGGCACCGAGTCGGTGCTTTT
sgRNA 2 (SEQ ID NO: 44):
GGACGTGGAGCTGGCCGAGGGTTTTAGAGCTAGAAATAGCAAGTTAAAAT
AAGGCTAGTCCGTTATCAACTTGAAAAAGTGGCACCGAGTCGGTGCTTTT
sgRNA 3 (SEQ ID NO: 45):
GAGGCGCTCCCCAAGAAGACGTTTTAGAGCTAGAAATAGCAAGTTAAAAT
AAGGCTAGTCCGTTATCAACTTGAAAAAGTGGCACCGAGTCGGTGCTTTT
sgRNA 4 (SEQ ID NO: 46):
GGGGGCCCCAGGGCTCCAGGGTTTTAGAGCTAGAAATAGCAAGTTAAAAT
AAGGCTAGTCCGTTATCAACTTGAAAAAGTGGCACCGAGTCGGTGCTTTT
sgRNA 5 (SEQ ID NO: 47):
GCTGAGGAACAAGCACCGCCGTTTTAGAGCTAGAAATAGCAAGTTAAAAT
AAGGCTAGTCCGTTATCAACTTGAAAAAGTGGCACCGAGTCGGTGCTTTT
sgRNA 6 (SEQ ID NO: 48):
GGCGCCTGCCACGATCAGGAGTTTTAGAGCTAGAAATAGCAAGTTAAAAT
AAGGCTAGTCCGTTATCAACTTGAAAAAGTGGCACCGAGTCGGTGCTTTT
sgRNA 7 (SEQ ID NO: 49):
GTGCAGCAGGCAGAAGAGCGGTTTTAGAGCTAGAAATAGCAAGTTAAAAT
AAGGCTAGTCCGTTATCAACTTGAAAAAGTGGCACCGAGTCGGTGCTTTT
sgRNA 8 (SEQ ID NO: 50):
GGAGTGATCGGCCCCCAGAGTTTTAGAGCTAGAAATAGCAAGTTAAAATA
AGGCTAGTCCGTTATCAACTTGAAAAAGTGGCACCGAGTCGGTGCTTTT

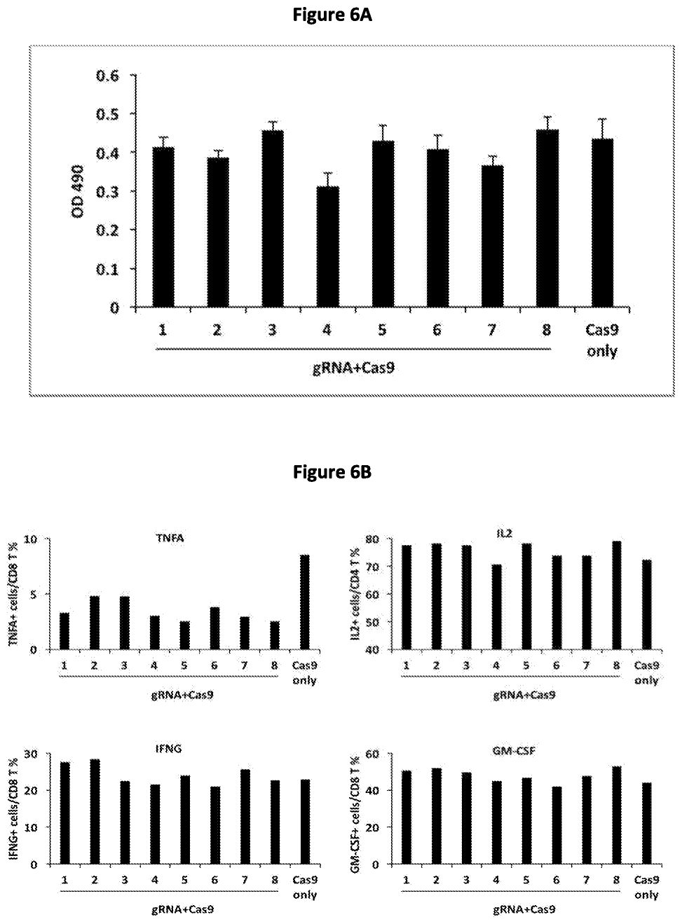

Five days after electroporation, T cells were assessed for proliferation using the CellTiter 96® AQueous One Solution Cell Proliferation Assay (MTS) (Promega). The results suggest that gene editing against TNFA did not significantly alter T cell proliferation after electroporation in the presence of exogenous IL2. FIG. 6A.

The T cells were activated with PMA/Ionomycin to analyze cytokine expression (GM-CSF, IL-2, IFNγ, and TNFα) through intracellular staining kit (Biolegend and BD Bioscience). The results indicate that the TNFA gene editing significantly reduced TNFα expression but showed no significant impact on GM-CSF, IFNγ or IL2 expression. FIG. 6B.

### Example 7: GM-CSF Knock-Out and IL6 Blocker/IL-1 Blocker-Secreting Anti-CD19 CART Cells Exerted Effective Cytotoxicity and IL6 and IL1B Inhibitory Effect

Primary T cells from healthy donor (PPA research) were activated by anti-CD3/CD28 beads (Thermo scientific). One day later, the T cells were transduced with a lentiviral vector encoding (i) an anti-CD19 CAR, and (ii) an anti-IL6 scFv polypeptide (SEQ ID NO:14), which has a V_{H} sequence of SEQ ID NO:3 and a V_{L} sequence of SEQ ID NO:4, and (iii) IL1RA.

The anti-CD19 CAR contains, from the N-terminus to the C-terminus, a CD8 leading sequence, an anti-CD19 scFv fragment, a CD8 hinge domain, a CD8 transmembrane domain, a 4-1BB co-stimulatory domain, and a CD3ζ domain. Exemplary amino acid sequences of these domains are provided below:

CD8 leading sequence (SEQ ID NO: 51):
MALPVTALLLPLALLLHAARP
Anti-CD19 scFv (SEQ ID NO: 52):
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTEGG
GTKLEITGGGGSGGGGSGGGGSEVKLQESGPGLVAPSQSLSVTCTVSGVS
LPDYGVSWIRQPPRKGLEWLGVINGSETTYYNSALKSRLTIIKDNSKSQV
FLKMNSLQTDDTAIYYCAKHYYYGGSYAMDYWGQGTSVTVSS
CD8 hinge domain (SEQ ID NO: 53):
TTTPAPRPPTPAPTIASQPLSLRPEACRPAAGGAVHTRGLDFACD
CD8 Transmembranedomain (SEQ ID NO: 54):
IYIWAPLAGTCGVLLLSLVITLYC
4-1BB co-stimulatory domain (SEQ ID NO: 55):
KRGRKKLLYIFKQPFMRPVQTTQEEDGCSCRFPEEEEGGCEL
CD3z (SEQ ID NO: 56):
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

A CD8 leading sequence is located before the anti-IL6 scFv. A nucleotide sequence coding for a T2A peptide is located between the coding sequences of (i) and (ii) and a nucleotide sequence coding for a P2A peptide is located between the coding sequences of (ii) and (iii). There is a human growth hormone signal sequence located between P2A and (iii). The resultant engineered T cells express the anti-CD19 CAR and secrets the anti-IL6 scFv antibody and IL-1RA (anti-CD19/IL6/IL1). Two days later, the T cells were electroporated with a Cas9 protein (Thermo scientific) together with a gRNA targeting the first exon of GM-CSF and optionally a gRNA targeting the TCR beta chain constant region. The resulting anti-CD19 CART cells were either anti-CD19/IL6/IL1 TCR⁻ or anti-CD19/IL6/IL1 TCR⁻/GM-CSF⁻. These cells were expanded and tested for CD3 expression by FACS analysis.

Expression of the anti-CD19 CAR was analyzed by a primary biotinylated goat-anti-mouse IgG-F(ab′)2 fragment followed by a secondary staining using Streptavidin conjugated with R-Phycoerythrin. There was 81.7% anti-CD19/IL6/IL1 TCR⁻ cells that were CD45+/CD3− and 78.9% anti-CD19/IL6/IL1 TCR⁻/GM-CSF⁻ cells that were CD45+/CD3−. The CD45+/CD3− cell population were then analyzed for CD4 and anti-CD19 CAR expression. There was about 10.7% anti-CD19 CART cells in CD45+/CD3− aCD19-61 TCR-cells, and 13.1% anti-CD19 CART cells in CD45+/CD3− aCD19-61 TCR−/GM-CSF− cells. Lastly, the CART cells were analyzed for their CD4/CD8 expression. In anti-CD19/IL6/IL1 TCR⁻ cells, there was about 12.1% CD8+ T cells and 85.3% CD4+ T cells. In anti-CD19/IL6/IL1 TCR⁻/GM-CSF⁻ cells, there was 13.3% CD8+ T cells and 81.2% CD4+ T cells.

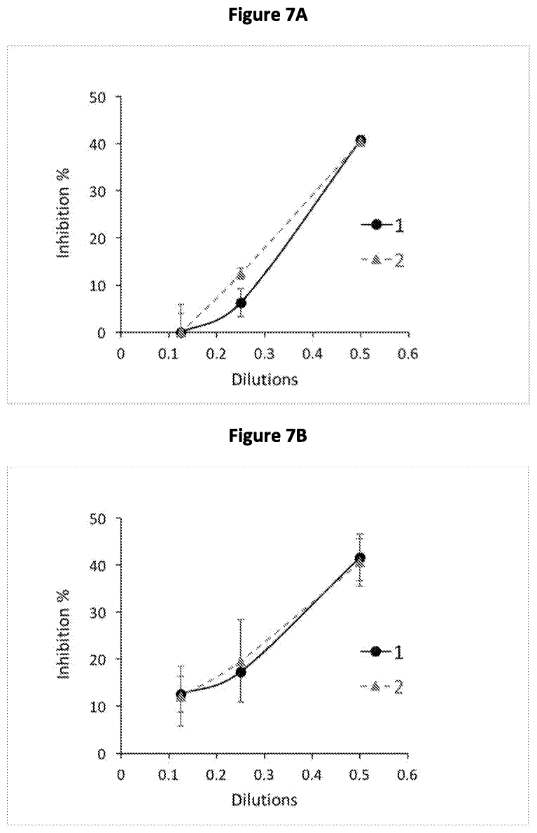

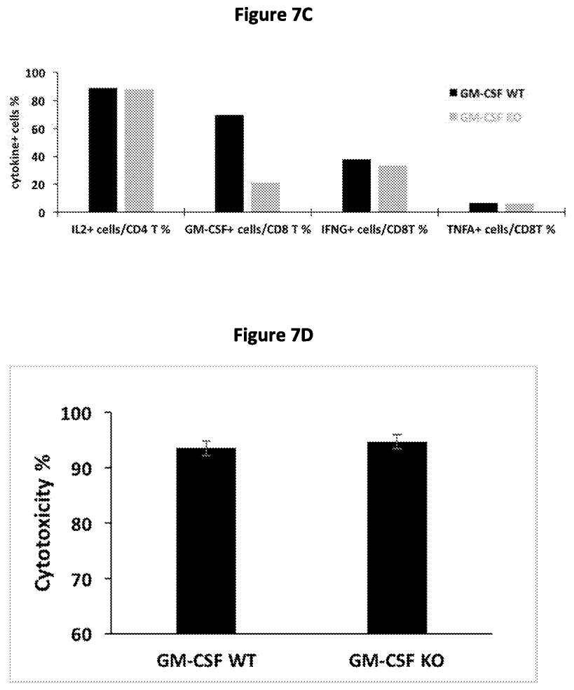

Anti-CD19/IL6/IL1 TCR− cells and anti-CD19/IL6/IL1 TCR⁻/GM-CSF⁻ cells were cultured with Nalm6-GFP cells at 1:1 E:T ratio for 2 days. The supernatant was collected from both co-cultures and added to HEK-Blue IL-6 reporter cells (Invivogen) in the presence of 1 ng/ml human IL-6 or HEK-Blue IL1R reporter cells in the presence of 5 pg/ml human IL1B. After overnight incubation, the supernatant of HEK-Blue IL-6 cells or HEK-Blue IL1R cells were collected and incubated with Quant-Blue substrate solution. SEAP production was quantified by measuring optical absorbance of converted substrate Quant Blue (Invivogen) at 650 nm wavelength through a spectrophotometer. As shown in FIG. 7A, supernatant from both anti-CD19/IL6/IL1 TCR⁻ cells and anti-CD19/IL6/IL1 TCR⁻/GM-CSF⁻ cells were able to inhibit IL6 signaling at higher than 0.1 dilution. As shown in FIG. 7B, supernatant from both anti-CD19/IL6/IL1 TCR− cells and anti-CD19/IL6/IL1 TCR⁻/GM-CSF⁻ cells were able to inhibit IL1B signaling at higher than 0.1 dilution.

Anti-CD19/IL6/IL1 TCR⁻ and anti-CD19/IL6/IL1 TCR⁻/GM-CSF⁻ cells were then activated by PMA/Ionomycin and tested for their cytokine expression (IL2, GM-CSF, IFNγ and TNFA). FIG. 7C shows the percentage of T cells that can secrete IL2, IFNγ, and TNFα were similar in both cell populations while there were a lot less GM-CSF secreting T cells in the anti-CD19/IL6/IL1 TCR⁻/GM-CSF⁻ cell population compared to anti-CD19/IL6/IL1 TCR⁻ cells.

The ability of anti-CD19/IL6/IL1 TCR⁻ cells and anti-CD19/IL6/IL1 TCR⁻/GM-CSF⁻ cells in killing CD19+ Nalm6 cells were evaluated. After the co-culture, the remaining number of tumor cells were analyzed by BD turcount beads and the percent of cytotoxicity was graphed in FIG. 7D. Both anti-CD19/IL6/IL1 TCR⁻ cells and anti-CD19/IL6/IL1 TCR⁻ /GM-CSF⁻ cells showed higher than 90% cytotoxicity against Nalm6 cells.

### Example 8: GM-CSF Knock-Out and IL6 Blocker/IL-1 Blocker-Secreting Anti-BCMA CAR-T Cells Exert Effective Cytotoxicity and IL6 and IL1B Inhibitory Effect

Primary T cells from healthy donor (PPA research) were activated by anti-CD3/CD28 beads (Thermo scientific). One day later, the T cells were transduced with a lentiviral vector encoding (i) an anti-BCMA CAR, and (ii) an anti-IL6 scFv polypeptide (SEQ ID NO:14), having a V_{H} sequence of SEQ ID NO:3 and a V_{L} sequence of SEQ ID NO:4, and (iii) IL1RA.

The anti-BCMA CAR contains, from the N-terminus to the C-terminus, a CD8 leading sequence, an anti-BCMA scFv fragment, a CD8 hinge domain, a CD8 transmembrane domain, a 4-1BB co-stimulatory domain, and a CD3ζ domain. Sequences for the CD8 leading sequence, the CD8 hinge and transmembrane domains, the 4-1BB co-stimulatory domain, and CD3ζ are provided in Example 7 above. The amino acid sequence of the anti-BCMA scFv is provided below (SEQ ID NO:57):

DIVLTQSPPSLAMSLGKRATISCRASESVTILGSHLIHWYQQKPGQPPTL
LIQLASNVQTGVPARFSGSGSRTDFTLTIDPVEEDDVAVYYCLQSRTIPR
TFGGGTKLEIKGSTSGSGKPGSGEGSTKGQIQLVQSGPELKKPGETVKIS
CKASGYTFTDYSINWVKRAPGKGLKWMGWINTETREPAYAYDERGRFAFS
LETSASTAYLQINNLKYEDTATYFCALDYSYAMDYWGQGTSVTVSS

A CD8 leading sequence is located before the anti-IL6 scFv. A nucleotide sequence coding for a T2A peptide is located between the coding sequences of (i) and (ii) and a nucleotide sequence coding for a P2A peptide is located between the coding sequences of (ii) and (iii). There is a human growth hormone signal sequence located between P2A and (iii). Two days later, the T cells were electroporated with Cas9 protein (Thermo scientific) plus gRNA targeting the first exon of GM-CSF. These cells were expanded and tested for CD3 expression by FACS analysis and CD3+ population was gated for further analysis. Expression of anti-BCMA CAR was analyzed by a primary biotinylated goat-anti-mouse IgG-F(ab′)2 fragment followed by a secondary staining using Streptavidin conjugated with R-Phycoerythrin. There was 98.9% CD3+ cells. The CD3+ cell population were then analyzed for CD4 and anti-BCMA CAR expression. There were about 5.78% anti-BCMA CAR-T cells in the CD3+ cell population. Lastly, the CAR-T cells were analyzed for their CD4/CD8 expression. There was about 23.8% CD8+ T cells and 73.6% CD4+ T cells.

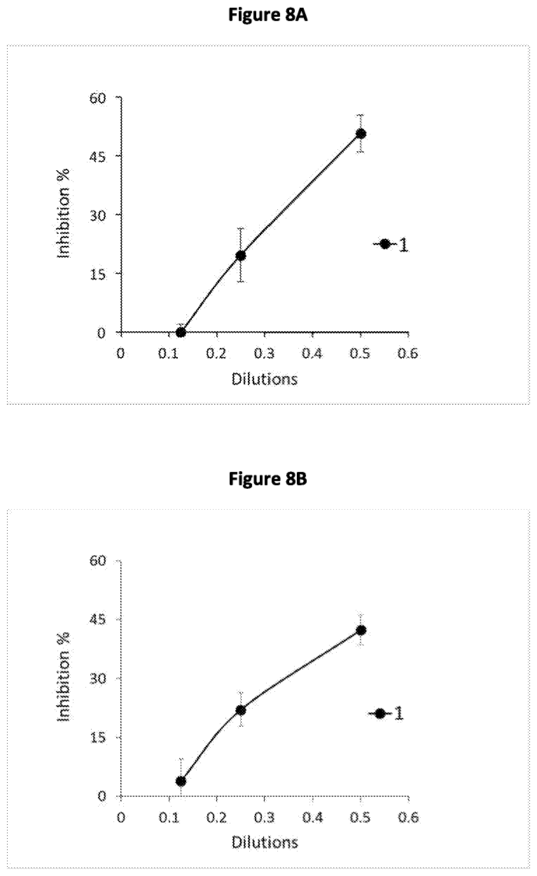

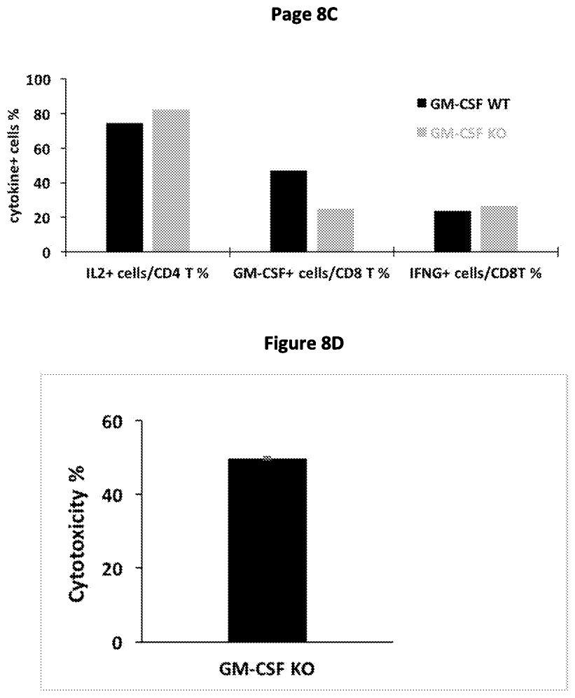

The anti-BCMA/IL6/IL1GM-CSF⁻ cells were cultured with RPMI-8226 cells at 1:1 E:T ratio for 2 days. The supernatant was collected from the co-culture and added to HEK-Blue IL-6 reporter cells (Invivogen) in the presence of 1 ng/ml human IL-6 or HEK-Blue IL1R reporter cells in the presence of 5 pg/ml human IL1B. After overnight incubation, the supernatant of HEK-Blue IL-6 cells or HEK-Blue IL1R cells were collected and incubated with Quant-Blue substrate solution. SEAP production was quantified by measuring optical absorbance of converted substrate Quant Blue (Invivogen) at 650 nm wavelength through a spectrophotometer. As shown in FIG. 8A, supernatant from both Anti-BCMA/IL6/IL1GM-CSF⁻ cells was able to inhibit IL6 signaling at higher than 0.1 dilution. As shown in FIG. 8B, supernatant from Anti-BCMA/IL6/IL1 GM-CSF⁻ cells was able to inhibit IL1B signaling at higher than 0.1 dilution.

The anti-BCMA/IL6/IL1 cells with or without disrupted GM-CSF were then tested for their cytokine expression (IL2, GM-CSF, and IFNγ), FIG. 8C shows the percentage of T cells that can secrete IL2, and IFNγ were similar in both cell populations while there were a lot less GM-CSF secreting T cells in the GM-CSF knock out cells compared to GM-CSF WT cells.

The ability of anti-BCMA/IL6/IL1/GM-CSF KO cells in killing BCMA+ RMPI-8226 cells was evaluated. After the co-culture, the remaining number of tumor cells was analyzed by BD turcount beads and the percent of cytotoxicity was graphed in FIG. 8D. The anti-BCMA/IL6/IL1/GM-CSF KO cells showed higher than 40% cytotoxicity against RPMI-8226 cells.

### Example 9: Therapeutic Effects of Anti-CD19/IL6/IL1/GM-CSF KO or Anti-CD19/IL6/IL1 CAR-T Cells in Human Cancer Patients

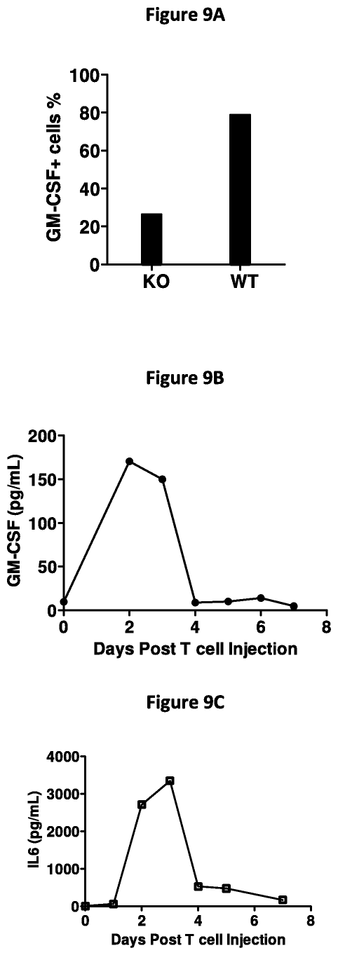

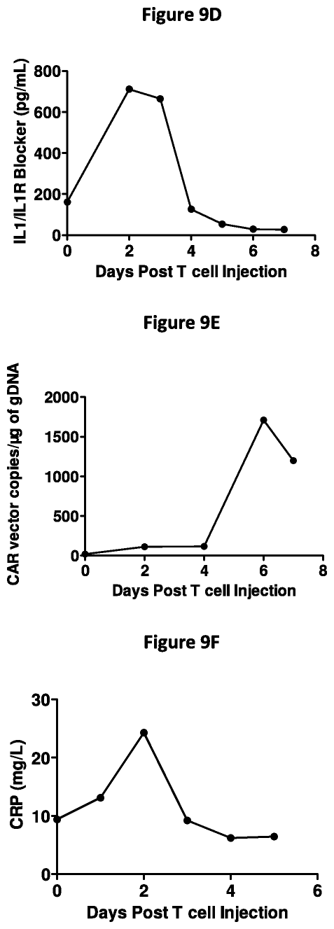

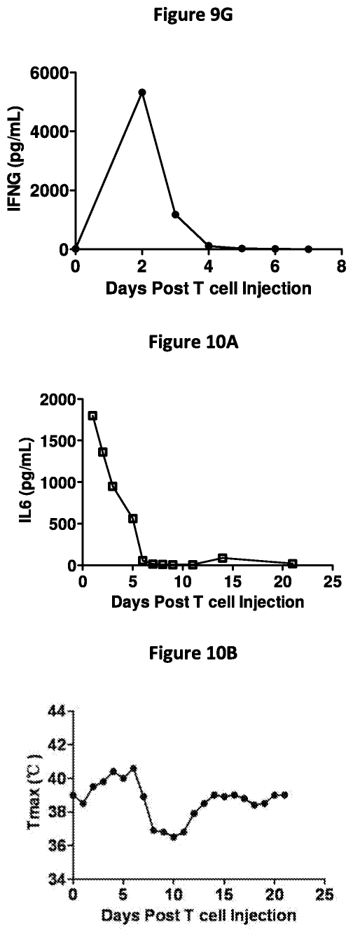

A human patient diagnosed for lymphoblastic leukemia (BCR/ABL1 fusion gene and ABL1 gene with T315I and E255K mutations) was subject to two rounds of treatment with anti-CD19/IL6/IL1/GM-CSF CAR-T cells described below. As shown in FIG. 9A, expression of GM-CSF in the CAR-T cells was substantially reduced as compared with wild-type counterparts.

### First Treatment

The human patient was first treated with chemo-therapy to lower tumor burden, followed by fludarabine/cyclophosphamide pretreatment to deplete endogenous lymphocytes so as to place the patient in condition for CAR-T cell transplantation. After the chemotherapy and the pretreatment, the total lymphocyte count (reflecting tumor burden) decreased to 0.03×10⁹/L. Afterwards, the patient received 1×10⁸ anti-CD19/IL6/IL1/GM-CSF KO CAR-T cells as disclosed herein (e.g., Examples 7 and 8 above, except that the CAR-T cells used in this Example are TCR positive). 46.8% CD19+ cells were detected in the peripheral blood of the patient at 5 days after CAR-T cell infusion, as relative to 95% CD19+ cells before the chemotherapy. The total number of CD19+ cells rapidly increased to approximately 12.4×10⁹/L at 7 days after CAR-T cell infusion, indicating only partial response to the CAR-T treatment.

Tocilizumab was injected to the patient at around 8 hours after the CAR-T cell infusion because of high fever—the patient displayed grade 2-3 CRS of fever, hypotension and hypoxia. Signs of CRS happened very early at about 4 hours after the CAR-T cell infusion, suggesting that the tumor burden at the onset of the CAR-T treatment (1^{st}) was relatively heavy. Analysis of the cytokine levels in the patient by ELISA revealed a substantially reduced levels of GM-CSF (FIG. 9B), and a moderate level of IL1/IL1R blocker (FIG. 9D). These results confirm successful knocking-out of the GM-CSF gene in the CAR-T cells and successful expression of the IL1/IL1R blockers by the CAR-T cells. The maximum daily body temperature (Tmax, ° C.) of the patient is shown in FIG. 9H.

Quantification of CAR vector copies by qPCR suggested limited expansion of CAR-T cells in vivo. FIG. 9E. This may be a reason for rapid relapse of the CD19+ tumor cells in the patient. The level of C-reactive Protein (CRP) elevated after CAR-T cell infusion and reached the peak two days after the infusion. FIG. 9F. A high level of IFNγ was also observed at day 2 after the infusion. The level of IL6 is also decreased (FIG. 9C), which may be attributable to the decrease of CAR-T and/or IFNγ activity.

### Second Treatment

The patient was then treated with very strong chemo-therapy to lower tumor burden, followed by fludarabine/cyclophosphamide pretreatment to deplete lymphocytes. After the chemotherapy and the pretreatment, the total lymphocyte count (reflecting tumor burden) decreased to a level that is barely detectable by flowcytometry-based analysis. Afterwards, the patient received two consecutive doses of 0.3×10⁸ (on D0) and 1×10⁸ (on D1) anti-CD19/IL6/IL1 CAR-T cells (with a wild-type GM-CSF gene). The patient was also given IL-2 once or multiple times during the therapy. At D7 and 33 days after CART infusion, B cell aplasia was detected in peripheral blood, indicating complete response to the CAR-T treatment.

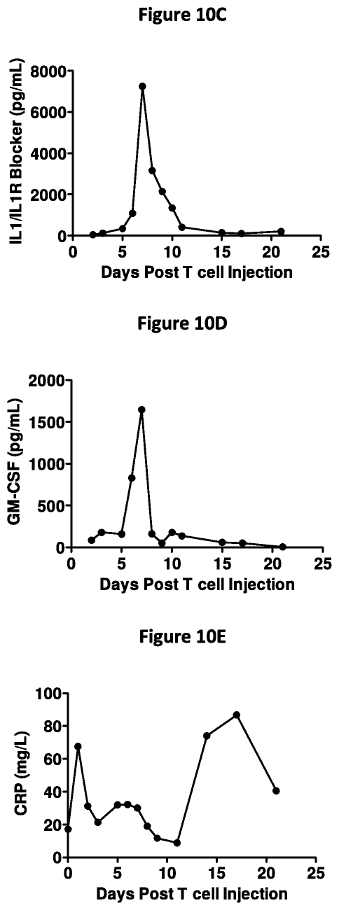

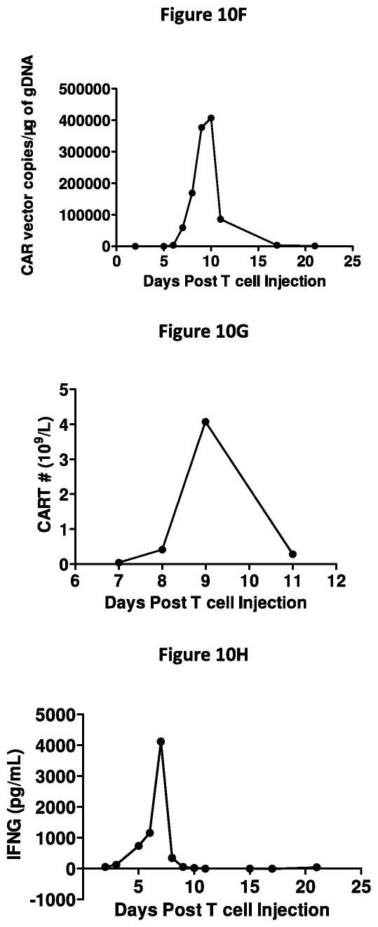

Analysis of cytokine level revealed a substantial decrease of IL-6 in the patient after the T cell infusion (FIG. 10A), a high levels of IL1/IL1R blocker, GM-CSF, and IFNγ during D5-D10 (FIG. 10C, FIG. 10D, and FIG. 10H, respectively).

The patient was diagnosed of infection after the pretreatment for lymph-depletion and before the T-cell infusion. Potential infection may be the cause of the high level of IL6 right after the infusion. However, the level of IL6 decreased substantially over time and reached the lowest point on the same time when the secretion of IL1/IL1R blocker reached the highest peak (FIG. 10A and FIG. 10C). The IL1/IL1R blocker is proportionally co-expressed with the IL6/IL6R blocker, coding sequences of the two blockers being linked by a nucleotide sequence encoding a P2A peptide linker. The maximum daily body temperatures (Tmax, ° C.) are shown in FIG. 10B.

Quantification of CAR vector copies by qPCR and analysis of CAR+ T cells by flow cytometry showed maximal expansion of CAR-T cells in vivo, resulting complete eradication of CD19+ tumor cells after CART treatment. FIG. 10F, and FIG. 10H. FIG. 10E shows the level of CRP after the T cell infusion.

From D0 to D21, the patient did not receive Tocilizumab and was only treated with ibuprofen, nasal cannula. The patient displayed only grade 1-2 CRS of fever and hypoxia. No symptom of neurotoxicity was observed in the patient.

In sum, these results indicated maximal expansion of CAR-T cells, maximal levels of cytokine secretion (IFNγ and GM-CSF), but extremely low level of IL6 during CRS peak time, and overall minimal to negligible symptoms of cytokine associated toxicity. See Table 1 below for CRS grading.

### Example 10: Therapeutic Effects of Anti-BCMA/IL6/IL1/GM-CSF/TCR KO CAR-T Cells in Human Refractory Multiple Myeloma Patients

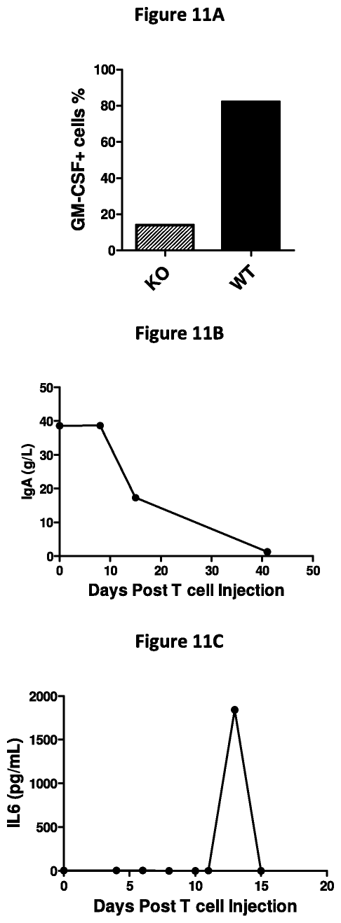

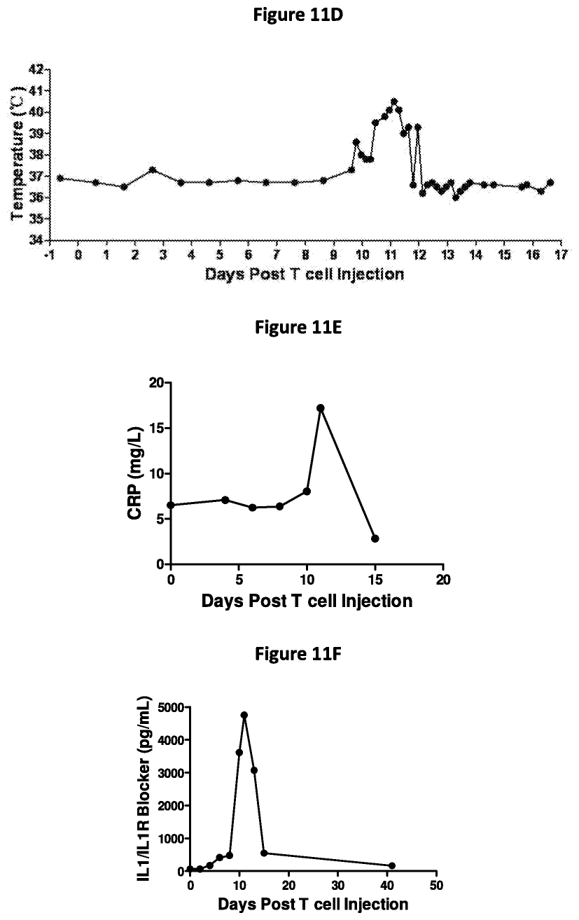

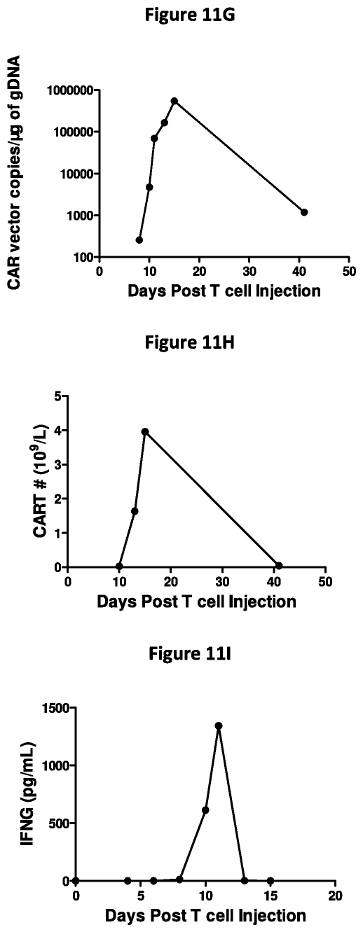

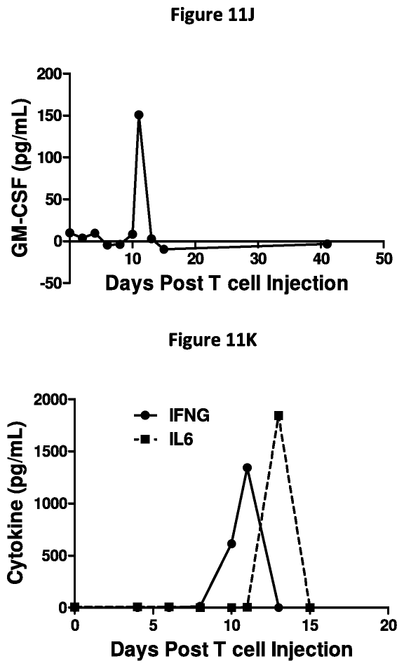

A refractory Multiple Myeloma (MM) patient was treated with a chemo-therapy to lower tumor burden, followed by Fludarabine/Cyclophosphamide pretreatment to deplete lymphocytes. Afterwards, the patient received two consecutive doses of 2×10⁶ (D0) and 3×10⁶ (D1) the anti-BCMA CART cells disclosed herein, which secrete IL6 and IL1 blockers and have the GM-CSF and TCR genes knocked out. The patient was also injected with human recombinant IL-2. The knock-out efficiency of the GM-CSF gene via CRISPR/Cas9 technology was shown in FIG. 11A. Crispr/Cas9 gene editing of TCR resulted in around 80% CD3− T cells, among which CD8 T cells were gated to analyze GM-CSF secreting cells by intracellular cytokine staining. The GM-CSF knock-out cell population contains less than 20% GM-CSF positive cells.

Before CAR-T cell infusion, the patient had around 13.9% BCMA⁺ plasma cells in the peripheral blood. The level of BCMA⁺ plasma cells in the peripheral blood decreased to 0.074% at Day 15 post CAR-T infusion, indicating the patient's complete response to the CAR-T treatment. In addition, the aberrant level of IgA decreased from 38.6 g/L before treatment to 1.25 g/L at D41 after the CAR-T treatment, indicating eradication of malignant plasma cells. FIG. 11B.

Serum levels of various cytokines in the patient were analyzed routine practice such as ELISA. The results showed a high level of IFNγ secretion and the IL1/IL1R blocker, but a low level of GM-CSF secretion. FIGS. 11F 11I and 11J. However, the level of IL6 was extremely low at the peak time point of IL1/IL1R blocker secretion, the synthesis of which is proportionally co-expressed with IL6/IL6R blocker by a P2A linker. FIG. 11C, FIG. 11F, and FIG. 11K. Quantification of CAR vector copies by qPCR and analysis of CAR+ T cells by flowcytometry suggest maximal expansion of CAR-T cells in vivo, resulting complete eradication of BCMA⁺ tumor cells after CART treatment. FIG. 11G. The numbers of CAR-T cells in the peripheral blood post CAR-T infusion are shown in FIG. 11H.

From D0 to D15, patient did not receive Tocilizumab and was only treated with ibuprofen, nasal cannula. The patient displayed only grade 1-2 CRS of fever and mild hypoxia as shown in Table 1. No neurotoxicity was observed.

TABLE 1
CRS in Patient Treated with Anti-BCMA CAR-T Cells

| CRS | | | | |
|---|---|---|---|---|
| Parameter | Grade 1 | Grade 2 | Grade 3 | Grade 4 |
| Fever | Temperature | Temperature | Temperature | Temperature |
| | ≥38° C. | ≥38° C. | ≥38° C. | ≥38° C. |

With either:

| | | | | |
|---|---|---|---|---|
| Hypo- | None | Not | Requiring one | Requiring |
| tension | | requiring | vasopressor | multiple |
| | | vasopressors | with or | vasopressors |
| | | | without | (excluding |
| | | | vasopressin | vasopressin) |

And/or:

| | | | | |
|---|---|---|---|---|
| Hypoxia | None | Requiring | Requiring | Requiring |
| | | low-flow | high-flow | positive |
| | | nasal | nasal | pressure (eg: |
| | | cannula or | cannula, | CPAP, |
| | | blow-by | facemask, | BiPAP, |
| | | | nonrebreather | intubation |
| | | | mask, or | and mechanical |
| | | | Venturi mask | ventilation) |

In sum, these results indicated maximal expansion of the anti-BCMA CAR-T cells in the patient having refractory multiple myeloma, maximal level of cytokine secretion (IFNγ), but extremely low level of IL6 during the CRS peak time (from D9 to D11), overall minimal to negligible symptoms of cytokine release syndrome, and none neurotoxicity. Interestingly, when the concentration of IL6/IL6R blocker decreased gradually after the peak time D11, IL6 level increased to a very high level at D13 transiently, which did not cause any fever or other toxicity.

### Example 11: Therapeutic Effects of Anti-CD19/IL6/IL1TCR Knock-Out CAR-T Cells with or without GM-CSF Knock-Out in Xenograft Mice

6-8 weeks old NSG mice were intravenously injected with 1×10⁶ Nalm6 leukemia cells modified to stably express GFP. 6 days later, the mice were intravenously injected with 2×10⁶TCR KO anti-CD19 CAR-T cells, which also express IL6/IL6R blocker and IL1/IL1R blocker with GM-CSF WT (indicated “1” in figures, n=5) or KO (indicated “2” in the figures, n=6). Mice not receiving CAR-T cells were included as controls (indicated “CTRL” in the figures, n=4). Post T cells injection, the mice were monitored for body weight, survival, and the number of GFP+ Nalm6 leukemia cells and CD45+CD3− T cells in the blood by Trucount beads (BD Biosciences).

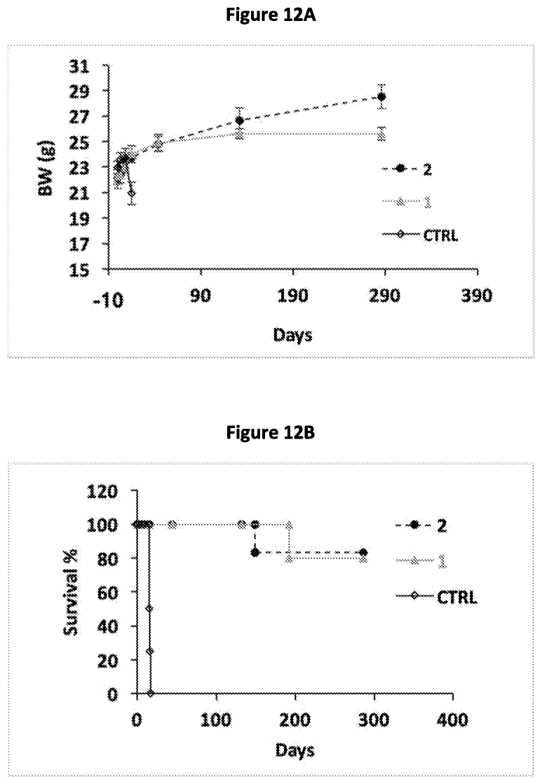

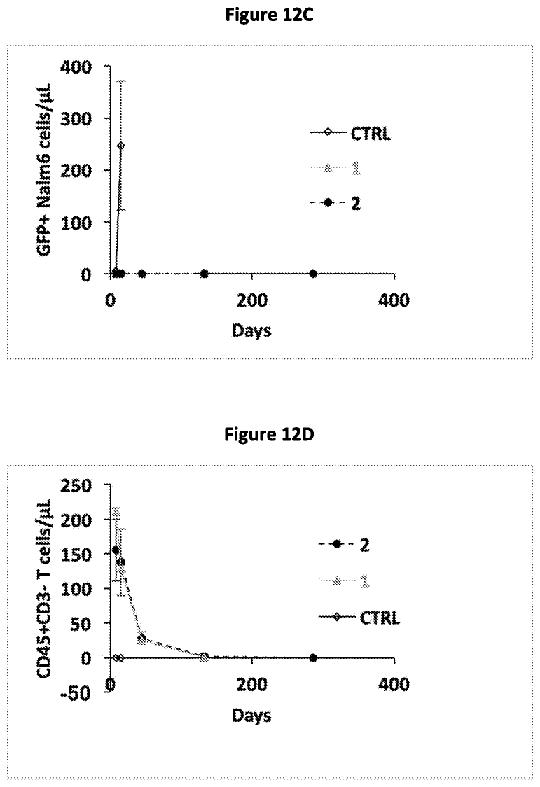

Body weights of the treated mice were monitored before and after the CAR-T cell treatment. Mice treated with anti-CD19 CAR-T cells, with or without GM-CSF KO, showed increase of body weights over time, while the body weights of the control mice dropped significantly. FIG. 12A. The anti-CD19 CAR-T cells, with or without GM-CSF KO, significantly prolonged survival rates of the treated mice and reduced the level of leukemia cells as relative to the control mice. FIGS. 12B and 12C. Finally, the numbers of T cells in the mice treated with the CAR-T cells decreased over time. FIG. 12D.

Overall, the CAR-T cells effectively eradicated leukemia cells in mice, maintained long term survival. Also, the cytokine KO T cells in the treated mice decreased gradually and did not transform to tumor like cells, suggesting safety of the CAR-T cells disclosed herein.

### Example 12: Therapeutic Effects of Anti-CD19/IL6/IL1/GM-CSF/TCR KO CAR-T Cells in Human Non-Hodgkin Lymphoma Patients

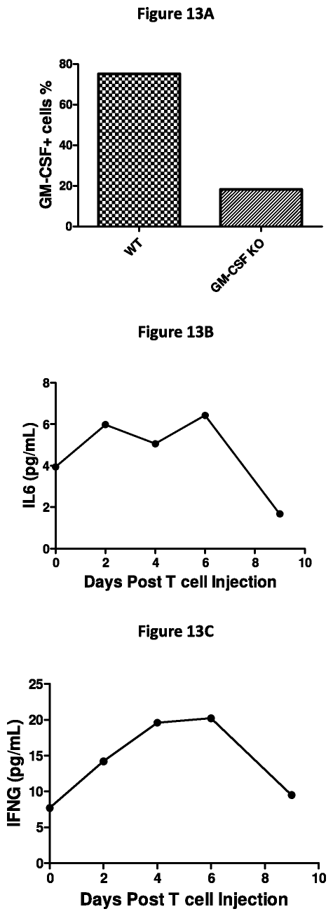

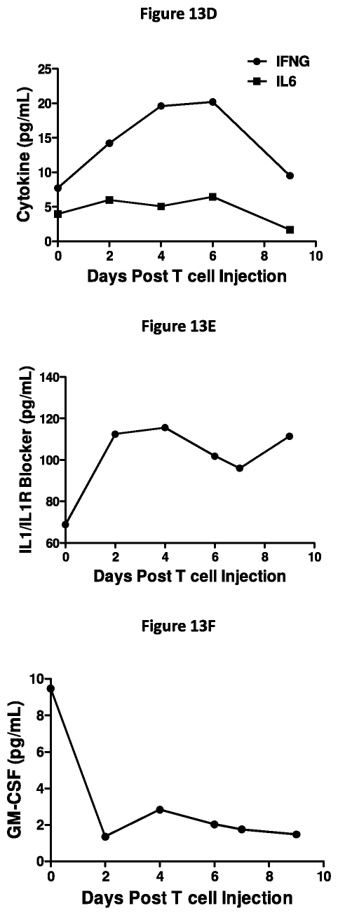

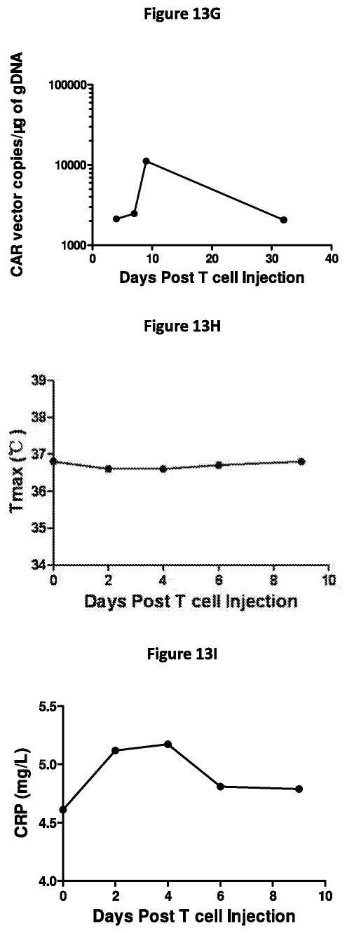

A human patient diagnosed for non-Hodgkin lymphoma was subject to treatment with the anti-CD19/IL6/IL1/TCR/GM-CSF KO CAR-T cells disclosed herein as follows. The human patient was treated with chemo-therapy to lower tumor burden, followed by fludarabine/cyclophosphamide pretreatment to deplete endogenous lymphocytes so as to place the patient in condition for CAR-T cell transplantation. Afterwards, the patient received 0.2×10⁸ (on day 0, D0) and 0.3×10⁸ (on day 1, D1) the anti-CD19/IL6/IL1 CAR-T cells as disclosed herein (with GM-CSF and TCR genes knocked out). As shown in FIG. 13A, a substantial portion of the T cells have GM-CSF knocked-out. The result showed the Crispr/Cas9 gene editing efficiency of GM-CSF KO (FIG. 13A). The patient was injected with recombinant IL2 during the therapy. At Day 7 post CART infusion, B cell aplasia was detected by flowcytometry analysis, indicating complete response to the CAR-T therapy.

Analysis of cytokine levels revealed a low level of IL-6 in the patient after the T cell infusion (FIGS. 13B and 13D), and slight increase of IL1/IL1R blocker, GM-CSF, and IFNG (FIG. 13E, FIG. 13F, and FIG. 13C, respectively). Quantification of CAR vector copies by qPCR showed significant expansion of CAR-T cells in vivo (FIG. 13G), which led to complete eradication of CD19⁺ tumor cells after the CAR-T treatment. FIG. 13I shows the level of CRP after the T cell infusion. From D0 to D9, the patient did not receive Tocilizumab, and displayed no symptoms of fever, hypoxia or hypotension. Furthermore, no neurotoxicity was detected during the treatment.

### Example 13: Therapeutic Effects of Anti-CD19/IL6/IL1 CAR-T Cells in Human Acute Lymphoblastic Leukemia Patients

A human patient diagnosed for acute lymphoblastic leukemia was subject to treatment with the anti-CD19/IL6/IL1 CAR-T cells disclosed herein as follows. The human patient was treated with chemo-therapy to lower tumor burden, followed by fludarabine/cyclophosphamide pretreatment to deplete endogenous lymphocytes so as to place the patient in condition for CAR-T cell transplantation. Afterwards, the patient received 0.35×10⁸ anti-CD19/IL6/IL1 CAR-T cells as disclosed herein (with wild-type GM-CSF and TCR genes). The patient was injected with recombinant IL2 during the therapy. At Day 14 post CAR-T cell infusion, the patient was diagnosed as negative of Minimal Residual Disease (MRD−), indicating complete response to the CAR-T therapy.

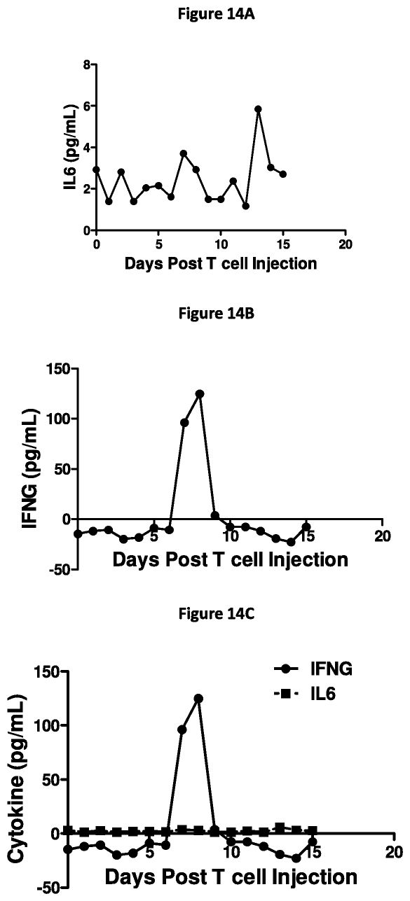

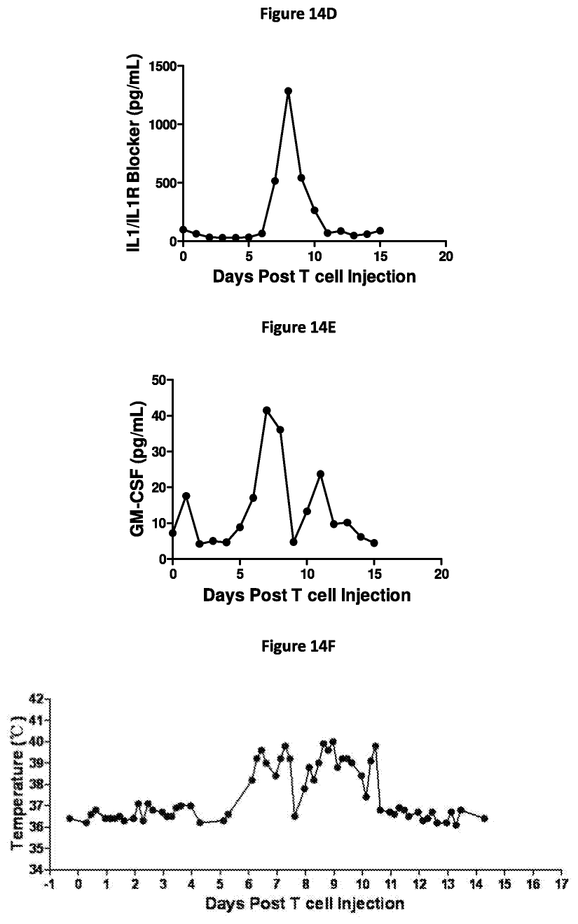

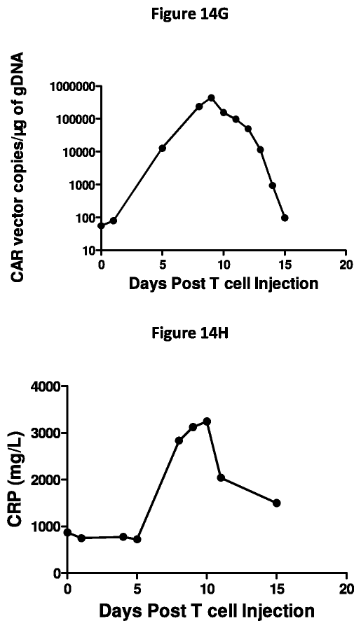

Analysis of cytokine levels revealed a low level of IL-6 in the patient after the T cell infusion (FIGS. 14A and 14C), a high level of IL1/IL1R blocker, GM-CSF, and IFNG (FIG. 14D, FIG. 14E, and FIG. 14B, respectively). Quantification of CAR vector copies by qPCR showed maximal expansion of CAR-T cells in vivo (FIG. 14G), leading to complete eradication of CD19⁺ tumor cells after the CAR-T treatment. FIG. 14H shows the level of CRP after the T cell infusion. From D0 to D15, the patient did not receive Tocilizumab, and displayed only grade 1 CRS of fever (FIG. 14F) without symptoms of hypoxia or hypotension. Furthermore, no neurotoxicity was detected during the treatment.

In sum, these results indicated maximal expansion of CAR-T cells, maximal levels of cytokine secretion (IFNG), but extremely low level of IL6 during CRS peak time, and overall minimal to negligible symptoms of cytokine associated toxicity, and no neurotoxicity.

### Example 14: Therapeutic Effects of Anti-BCMA/IL6/IL1 CAR-T Cells in Human Multiple Myeloma Patients

A Multiple Myeloma (MM) patient was treated with chemo-therapy to lower tumor burden, followed by fludarabine/cyclophosphamide pretreatment to deplete lymphocytes. Afterwards, the patient received a single dose of 4×10⁷ (D0) anti-BCMA CART cells secreting IL6/IL1 blockers with wild type GM-CSF and TCR genes, while the patient was also injected with human recombinant IL2. Throughout the treatment, the patient did not receive tocilizumab.

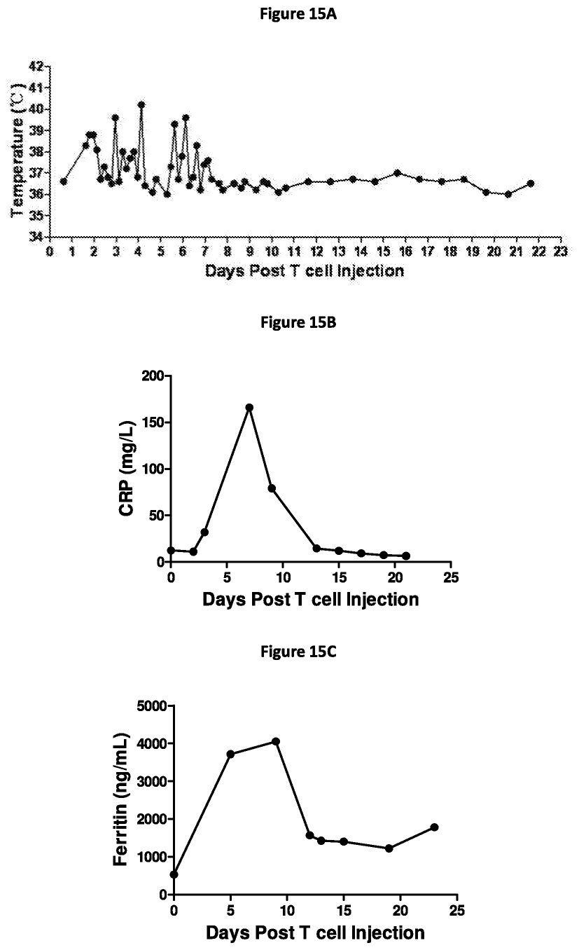

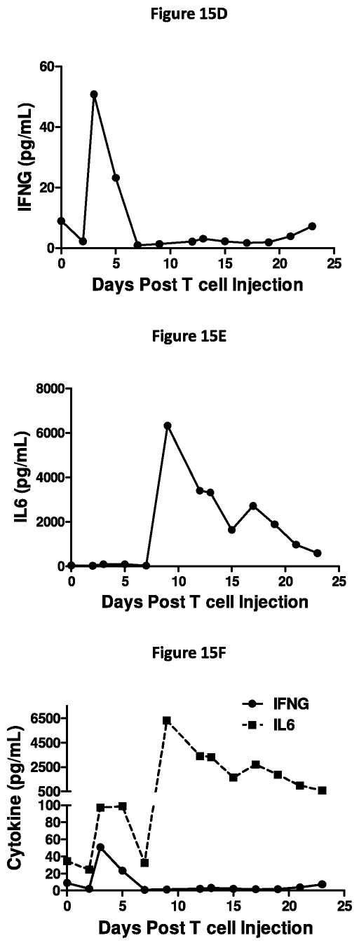

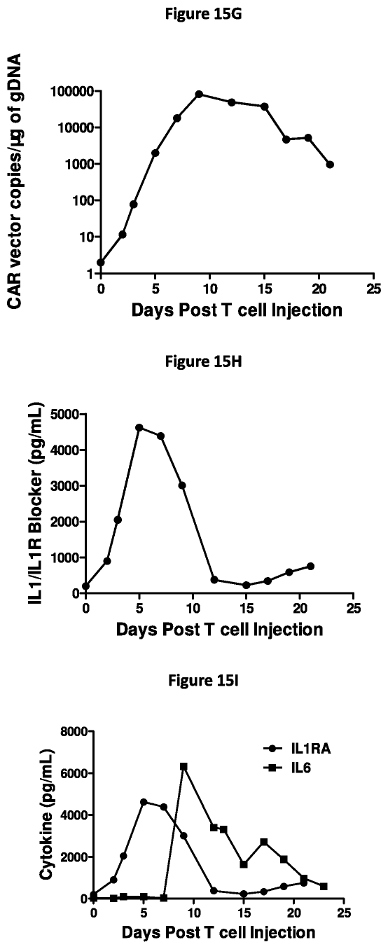

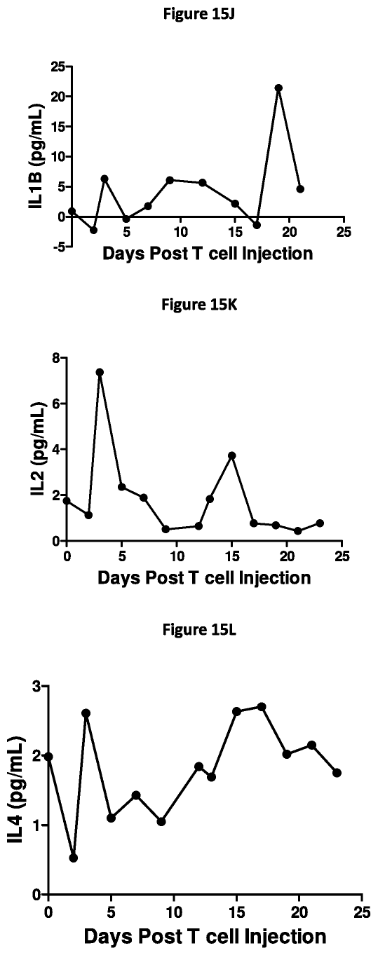

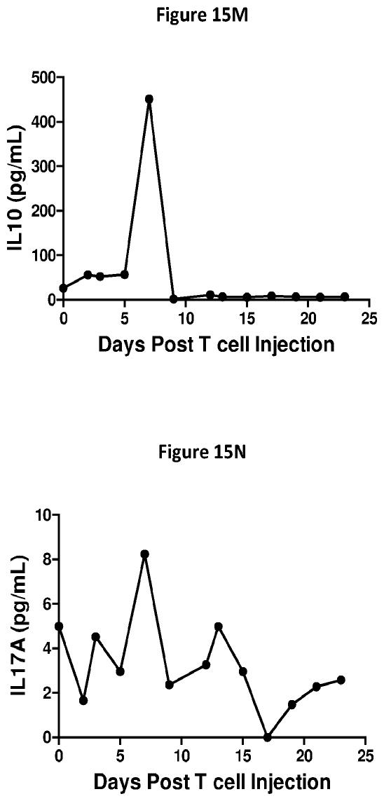

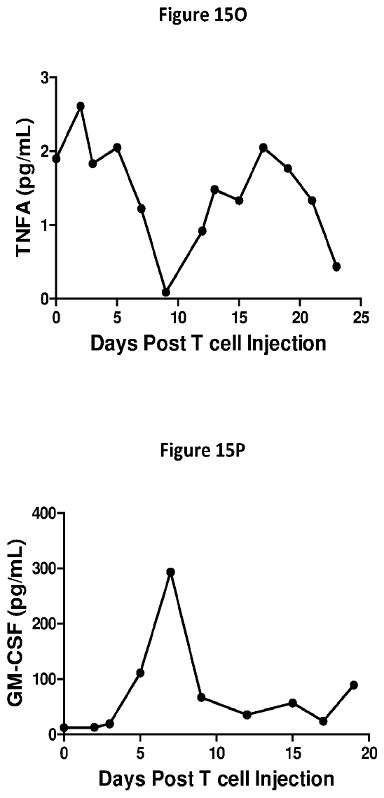

Monitoring of temperature (FIG. 15A) indicated fever from D1 to D6, along with increase of CRP, Ferritin and IFNG levels (FIGS. 15A-15D). However, IL6 level was maintained at low levels (<100 pg/mL, FIG. 15E) from D1 to D6, along with only grade 1 CRS of fever (without hypoxia and hypotension) and no neurotoxicity was observed. Significant increase of IL1RA (FIG. 15H) was detected during treatment, consistent with CART expansion (FIG. 15G). Comparison of IL6 level with IFNG and IL1RA levels (FIGS. 15F, 15H and 15I) suggests that IL6 secretion was initially inhibited during CAR-T cell expansion. However, IL6 increased dramatically at D9 and maintained at high levels from D9 to D23. Mild neurotoxicity was only observed during D13 to D17, during which no fever, hypoxia or hypotension was observed. Analysis of cytokines revealed low levels of IFNG, IL1B, IL2, IL4, IL10, IL17A, TNFA and GM-CSF from D13 to D17, suggesting high level of IL6 alone might cause neurotoxicity (FIGS. 15D and 15J-15P).

In summary, fever was only observed during D1 to D6 and mild neurotoxicity was only observed during D13 to D17 in the patient. No hypoxia or hypotension was observed during the CART treatment. The results above suggest that the patient went through typical CRS from D1 to D6, which was effectively reduced by the IL6 blocker produced by the CAR-T cells. Afterwards, dramatic increase of IL6 at D9 and the presence of high level IL6 from D13 to D17 were probably due to the down regulation of the IL6 blocker produced by the CAR-T cells, which caused mild neurotoxicity, without fever, hypoxia or hypotension. IL6 associated neurotoxicity further supports that IL6 is not only an important target for reducing CRS severity, but also for neurotoxicity.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A population of immune cells, wherein the immune cells express a chimeric receptor antigen (CAR) and an antibody specific to interleukin-6 (IL-6) or IL-6 receptor (IL-6R), wherein the antibody comprises the same heavy chain complementarity determining domains (CDRs) and the same light chain CDRs as a reference antibody, and wherein the reference antibody comprises (a) a heavy chain variable domain (V_{H}) set forth as SEQ ID NO:1 and a light chain variable domain (V_{L}) set forth as SEQ ID NO:2, or (b) a V_{H} set forth as SEQ ID NO:3 and a V_{L} set forth as SEQ ID NO:4.

2. The population of immune cells of claim 1, wherein the antibody specific to IL-6 or IL-6R comprises the same V_{H} and the same V_{L} as the reference antibody.

3. The population of immune cells of claim 1, wherein the antibody specific to IL-6 or IL-6R is a single-chain antibody fragment (scFv).

4. The population of immune cells of claim 3, wherein the scFv comprises the amino acid sequence of SEQ ID NO:13 or SEQ ID NO: 14.

5. The population of immune cells of claim 1, wherein at least 10% of the cells express both the CAR and the antibody specific to IL-6 or IL-6R.

6. The population of immune cells of claim 5, wherein about 50-70% of the cells express both the CAR and the antibody specific to IL-6 or IL-6R.

7. The population of immune cells of claim 1, wherein the antibody specific to interleukin-6 (IL-6) or IL-6 receptor (IL-6R) is a fragment of a bi-specific antibody, which further comprises an antibody specific to granulocyte macrophage-colony stimulating factor (GM-CSF).

8. The population of immune cells of claim 7, wherein the antibody specific to GM-CSF comprises the same heavy chain complementarity determining domains (CDRs) and the same light chain CDRs as a reference antibody, and wherein the reference antibody comprises (a) a heavy chain variable domain (V_{H}) set forth as SEQ ID NO:21 and a light chain variable domain (V_{L}) set forth as SEQ ID NO:22.

9. The population of immune cells of claim 7, wherein the antibody specific to GM-CSF comprises a V_{H} set forth as SEQ ID NO:21 and a V_{L} set forth as SEQ ID NO:22.

10. The population of immune cells of claim 7, wherein the antibody specific to GM-CSF is a scFv antibody.

11. The population of immune cells of claim 10, wherein the antibody specific to GM-CSF is linked to the antibody specific to IL-6 or IL-6R via a peptide linker.

12. The population of immune cells of claim 11, wherein the peptide linker is GSGGSG.

13. The population of immune cells of claim 7, wherein the bispecific antibody comprises the amino acid sequence of SEQ ID NO:28.

14. The population of immune cells of claim 1, wherein the immune cells express an IL-1 antagonist.

15. The population of immune cells of claim 14, wherein the IL-1 antagonist is IL-1RA.

16. The population of immune cells of claim 1, wherein the immune cells comprise a disrupted endogenous IL-2 gene, a disrupted endogenous GM-CSF gene, a disrupted TNFA gene, or a combination thereof.

17. The population of immune cells of claim 1, wherein the immune cells are T-cells, NK cells, dendritic cells, macrophages, B cells, neutrophils, eosinophils, basophils, mast cells, myeloid-derived suppressor cells, mesenchymal stem cells, precursors thereof, or a combination thereof.

18. The population of immune cells of claim 1, wherein the immune cells are T cells, which do not express an endogenous T cell receptor.

19. The population of immune cells of claim 1, wherein the CAR comprises an extracellular domain specific to a pathologic antigen, a transmembrane domain, and a cytoplasmic domain comprising one or more signaling domains.

20. The population of immune cells of claim 19, wherein the one or more signaling domains comprise one or more co-stimulatory domains, a cytoplasmic signaling domain of CD3, or a combination thereof.

21. The population of immune cells of claim 20, wherein the one or more co-stimulatory domains are derived from a co-stimulatory protein selected from the group consisting of CD28, 4-1BB, CD27, OX40, and ICOS.

22. The population of immune cells of claim 1, wherein the CAR binds CD19 or BCMA.

23. The population of immune cells of claim 22, wherein the CAR binds CD19 and comprises an extracellular domain, which is a single-chain antibody fragment comprising the amino acid sequence of SEQ ID NO:52.

24. The population of immune cells of claim 22, wherein the CAR binds BCMA and comprises an extracellular domain, which is a single-chain antibody fragment comprising the amino acid sequence of SEQ ID NO:57.

25. The population of immune cells of claim 22, wherein the immune cells further express an IL-1 antagonist, which optionally is IL-1RA.

26. The population of immune cells of claim 22, wherein the immune cells have a disrupted endogenous GM-CSF gene, and/or a disrupted endogenous TCR gene.

27. The population of immune cells of claim 22, wherein the immune cells have wild-type endogenous GM-CSF and/or TCR genes.

28. An immune cell, which expresses a chimeric receptor antigen (CAR) and an antibody specific to interleukin-6 (IL-6) or IL-6 receptor (IL-6R), wherein the antibody comprises the same heavy chain complementarity determining domains (CDRs) and the same light chain CDRs as a reference antibody, and wherein the reference antibody comprises (a) a heavy chain variable domain (V_{H}) set forth as SEQ ID NO:1 and a light chain variable domain (V_{L}) set forth as SEQ ID NO:2, or (b) a V_{H} set forth as SEQ ID NO:3 and a V_{L} set forth as SEQ ID NO:4.

29. The immune cell of claim 28, wherein the antibody specific to IL-6 or IL-6R comprises (a) a heavy chain variable domain (VH) set forth as SEQ ID NO:1 and a light chain variable domain (VL) set forth as SEQ ID NO:2, or (b) a VH set forth as SEQ ID NO:3 and a VL set forth as SEQ ID NO:4.

30. The immune cell of claim 28, wherein the antibody specific to interleukin-6 (IL-6) or IL-6 receptor (IL-6R) is a fragment of a bi-specific antibody, which further comprises an antibody specific to granulocyte macrophage-colony stimulating factor (GM-CSF).

31. The immune cell of claim 30, wherein the antibody specific to GM-CSF comprises the same heavy chain complementarity determining domains (CDRs) and the same light chain CDRs as a reference antibody, and wherein the reference antibody comprises (a) a heavy chain variable domain (V_{H}) set forth as SEQ ID NO:21 and a light chain variable domain (V_{L}) set forth as SEQ ID NO:22.

32. The immune cell of claim 28, wherein the immune cells express an IL-1 antagonist, which optionally is IL-1RA

33. The immune cell of claim 28, wherein the immune cells comprise a disrupted endogenous IL-2 gene, a disrupted endogenous GM-CSF gene, a disrupted endogenous TNFA gene, or a combination thereof.

34. The immune cell of claim 28, wherein the immune cell is a T-cell or an NK cell, a dendritic cell, a macrophage, a B cell, a neutrophil, an eosinophil, a basophil, a mast cell, a myeloid-derived suppressor cell, mesenchymal stem cells, or a precursor thereof.

35. The immune cell of claim 34, wherein the immune cell is a T cell, which does not express an endogenous T cell receptor.

36. The immune cell of claim 28, wherein the CAR comprises an extracellular domain specific to a pathologic antigen, a transmembrane domain, and a cytoplasmic domain comprising one or more signaling domains.

37. The immune cell of claim 36, wherein the CAR binds CD19 or BCMA and the immune cell further express an IL-1 antagonist, which optionally is IL-1RA.

38. The immune cell of claim 37, wherein the immune cells have a disrupted endogenous GM-CSF gene, and/or a disrupted endogenous TCR gene.

39. The immune cell of claim 37 wherein the immune cells have wild-type endogenous GM-CSF and/or TCR genes.

40. A method of producing a population of modified immune cells with reduced inflammatory properties, the method comprising:
(i) providing a population of immune cells; and
(ii) introducing into the immune cells a first nucleic acid coding for a chimeric antigen receptor (CAR) comprises an extracellular domain specific to a pathologic antigen, a transmembrane domain, and a cytoplasmic domain comprising one or more signaling domains, and a second nucleic acid coding for an antibody specific to interleukin-6 (IL-6) or IL-6 receptor (IL-6R), wherein the antibody is set forth in claim 1;
wherein both the first nucleic acid and the second nucleic acid are in operable linkage to a promoter for expression of the CAR and the antibody in the immune cells.

41. The method of claim 40, wherein the antibody specific to interleukin-6 (IL-6) or IL-6 receptor (IL-6R) is a fragment of a bi-specific antibody, which further comprises an antibody specific to granulocyte macrophage-colony stimulating factor (GM-CSF).

42. The method of claim 41, wherein the antibody specific to GM-CSF comprises the same heavy chain complementarity determining domains (CDRs) and the same light chain CDRs as a reference antibody, and wherein the reference antibody comprises (a) a heavy chain variable domain (V_{H}) set forth as SEQ ID NO:21 and a light chain variable domain (V_{L}) set forth as SEQ ID NO:22.

43. The method of claim 40, further comprising introducing into the immune cells a third nucleic acid encoding an IL-1 antagonist, which optionally is IL-1RA.

44. The method of claim 43, wherein the second nucleic acid and the third nucleic acid are located on the same vector.

45. The method of claim 40, further comprising disrupting in the immune cells an endogenous IL-2 gene, an endogenous GM-CSF gene, an endogenous TNFA, an endogenous T cell receptor (TCR) gene, or a combination thereof.

46. The method of claim 40, wherein the endogenous GM-CSF gene and/or the TCR gene are not disrupted.

47. The method of claim 45, wherein the endogenous gene(s) is disrupted by a CRISPR gene editing system.

48. The method of claim 40, wherein the immune cells are T-cells or NK cells.

49. A method of cell therapy, comprising administering to a subject in need thereof a population of immune cells of claim 1.

50. The method of claim 49, wherein the subject is a human patient having cancer, an infectious disease, or an immune disorder.

51. The method of claim 50, wherein the subject is a human patient having a cancer, and wherein the human patient has subjected to a therapy against the cancer to reduce tumor burden.

52. The method of claim 51, wherein the therapy is a chemotherapy, an immunotherapy, a radiotherapy, or a surgery.

53. The method of claim 40, wherein the subject has a lymphodepleting treatment prior to the cell therapy to conditioning the subject for the cell therapy.

54. The method of claim 53, wherein the lymphodepleting treatment comprises administering to the subject fludarabine and/or cyclophosphamide.

55. The method of claim 49, wherein the immune cells expresses an anti-CD19 CAR and the subject is a human patient having lymphoblastic leukemia or non-Hodgkin lymphoma, and wherein optionally the lymphoblastic leukemia is acute lymphoblastic leukemia.

56. The method of claim 49, wherein the immune cells expresses an anti-BCMA CAR and the subject is a human patient having multiple myeloma, which optionally is relapsed or refractory multiple myeloma.

57. The method of claim 55, wherein the immune cells further express an IL-1 antagonist, which optionally is IL-1RA and has a disrupted endogenous GM-CSF gene and/or a disrupted endogenous TCR gene.

58. The method of claim 55, wherein the immune cells further express an IL-1 antagonist, which optionally is IL-1RA and has wild-type endogenous GM-CSF and/or TCR genes.

59. A bi-specific antibody, comprising a first antibody fragment specific to IL-6 and a second antibody fragment specific to GM-CSF.

60. The bi-specific antibody of claim 59, wherein the antibody fragment specific to IL-6 comprises the same heavy chain complementarity determining domains (CDRs) and the same light chain CDRs as a reference antibody, and wherein the reference antibody comprises (a) a heavy chain variable domain (V_{H}) set forth as SEQ ID NO:1 and a light chain variable domain (V_{L}) set forth as SEQ ID NO:2, or (b) a VH set forth as SEQ ID NO:3 and a VL set forth as SEQ ID NO:4.

61. The bi-specific antibody of claim 60, wherein the antibody fragment specific to IL-6 or IL-6R comprises the same V_{H} and the same V_{L} as the reference antibody.

62. The bi-specific antibody of claim 59, wherein the antibody fragment specific to IL-6 or IL-6R is a single-chain antibody fragment (scFv).

63. The bi-specific antibody of claim 62, wherein the scFv comprises the amino acid sequence of SEQ ID NO:13 or SEQ ID NO: 14.

64. The bi-specific antibody of claim 59, wherein the antibody fragment specific to GM-CSF comprises the same heavy chain complementarity determining domains (CDRs) and the same light chain CDRs as a reference antibody, and wherein the reference antibody comprises (a) a heavy chain variable domain (V_{H}) set forth as SEQ ID NO:21 and a light chain variable domain (V_{L}) set forth as SEQ ID NO:22.

65. The bi-specific antibody of claim 64, wherein the antibody fragment specific to GM-CSF comprises a V_{H} set forth as SEQ ID NO:21 and a V_{L} set forth as SEQ ID NO:22.

66. The bi-specific antibody of claim 64, wherein the antibody specific to GM-CSF is a scFv antibody.

67. The bi-specific antibody of claim 59, wherein the antibody specific to GM-CSF is linked to the antibody specific to IL-6 or IL-6R via a peptide linker.

68. The bi-specific antibody of claim 67, wherein the peptide linker is GSGGSG.

69. The bi-specific antibody of claim 68, wherein the bispecific antibody comprises the amino acid sequence of SEQ ID NO:28.

70. A nucleic acid, comprising a first nucleotide sequence encoding an antibody fragment specific to IL-6, a second nucleotide sequence encoding a self-cleaving peptide, and a third nucleic sequence encoding an IL-1 antagonist.

71. The nucleic acid of claim 70, wherein the first nucleotide sequence encodes a bi-specific antibody comprising the antibody fragment specific to IL-6 and an antibody fragment specific to GM-CSF.


SEQUENCE LISTING (60 sequences)

SEQ ID NO: 1 - protein, 116 aa, Artificial Sequence
  Synthetic Polypeptide
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 2 - protein, 107 aa, Artificial Sequence
  Synthetic Polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 3 - protein, 119 aa, Artificial Sequence
  Synthetic Polypeptide
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 4 - protein, 106 aa, Artificial Sequence
  Synthetic Polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 5 - protein, 119 aa, Artificial Sequence [sequence omitted]

SEQ ID NO: 6 - protein, 106 aa, Artificial Sequence [sequence omitted]

SEQ ID NO: 7 - protein, 119 aa, Artificial Sequence [sequence omitted]

SEQ ID NO: 8 - protein, 107 aa, Artificial Sequence [sequence omitted]

SEQ ID NO: 9 - protein, 120 aa, Artificial Sequence
  Synthetic Polypeptide
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 10 - protein, 110 aa, Artificial Sequence
  Synthetic Polypeptide
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 11 - protein, 120 aa, Artificial Sequence [sequence omitted]

SEQ ID NO: 12 - protein, 107 aa, Artificial Sequence [sequence omitted]

SEQ ID NO: 13 - protein, 238 aa, Artificial Sequence
  Synthetic Polypeptide
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKGGGGSGGGGSGGGGSEVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 14 - protein, 240 aa, Artificial Sequence
  Synthetic Polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 15 - protein, 240 aa, Artificial Sequence
  Synthetic Polypeptide
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKGGGGSGGGGSGGGGSEVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSS

SEQ ID NO: 16 - protein, 246 aa, Artificial Sequence
  Synthetic Polypeptide
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSGGGGSGGRASGGGGSGGGGSDIQMTQSPSSLSASVGDRVTITCRASQDISSYLNWYQQKPGKAPKLLIYYTSRLHSGVPSRFSGSGSGTDFTFTISSLQPEDIATYYCQQGNTLPYTFGQGTKVEIK

SEQ ID NO: 17 - protein, 119 aa, Artificial Sequence
  Synthetic Polypeptide
QVQLVQSGAEVKKPGASVKVSCKAFGYPFTDYLLHWVRQAPGQGLEWVGWLNPYSGDTNYAQKFQGRVTMTRDTSISTAYMELSRLRSDDTAVYYCTRTTLISVYFDYWGQGTMVTVSS

SEQ ID NO: 18 - protein, 107 aa, Artificial Sequence
  Synthetic Polypeptide
DIQMTQSPSSVSASVGDRVTIACRASQNIRNILNWYQQRPGKAPQLLIYAASNLQSGVPSRFSGSGSGTDFTLTINSLQPEDFATYYCQQSYSMPRTFGGGTKLEIK

SEQ ID NO: 19 - protein, 121 aa, Artificial Sequence
  Synthetic Polypeptide
EVQLVESGGGLVQPGGSLRLSCAASGFTFSRHWMHWLRQVPGKGPVWVSRINGAGTSITYADSVRGRFTISRDNANNTLFLQMNSLRADDTALYFCARANSVWFRGLFDYWGQGTPVTVSS

SEQ ID NO: 20 - protein, 107 aa, Artificial Sequence
  Synthetic Polypeptide
EIVLTQSPVTLSVSPGERVTLSCRASQSVSTNLAWYQQKLGQGPRLLIYGASTRATDIPARFSGSGSETEFTLTISSLQSEDFAVYYCQQYDKWPDTFGQGTKLEIK

SEQ ID NO: 21 - protein, 117 aa, Artificial Sequence [sequence omitted]

SEQ ID NO: 22 - protein, 106 aa, Artificial Sequence
  Synthetic Polypeptide
DIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 23 - protein, 241 aa, Artificial Sequence
  Synthetic Polypeptide
QVQLVQSGAEVKKPGASVKVSCKAFGYPFTDYLLHWVRQAPGQGLEWVGWLNPYSGDTNYAQKFQGRVTMTRDTSISTAYMELSRLRSDDTAVYYCTRTTLISVYFDYWGQGTMVTVSSGGSGGSGGSGGSGGSDIQMTQSPSSVSASVGDRVTIACRASQNIRNILNWYQQRPGKAPQLLIYAASNLQSGVPSRFSGSGSGTDFTLTINSLQPEDFATYYCQQSYSMPRTFGGGTKLEIK

SEQ ID NO: 24 - protein, 243 aa, Artificial Sequence
  Synthetic Polypeptide
EVQLVESGGGLVQPGGSLRLSCAASGFTFSRHWMHWLRQVPGKGPVWVSRINGAGTSITYADSVRGRFTISRDNANNTLFLQMNSLRADDTALYFCARANSVWFRGLFDYWGQGTPVTVSSGGSGGSGGSGGSGGSEIVLTQSPVTLSVSPGERVTLSCRASQSVSTNLAWYQQKLGQGPRLLIYGASTRATDIPARFSGSGSETEFTLTISSLQSEDFAVYYCQQYDKWPDTFGQGTKLEIK

SEQ ID NO: 25 - protein, 238 aa, Artificial Sequence
  Synthetic Polypeptide
QVQLVESGGGLVQPGGSLRLSCAASGFTFSSYWMNWVRQAPGKGLEWVSGIENKYAGGATYYAASVKGRFTISRDNSKNTLYLQMNSLRAEDTAVYYCARGFGTDFWGQGTLVTVSSGGSGGSGGSGGSGGSDIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 26 - protein, 487 aa, Artificial Sequence
  Synthetic Polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSGSGGSGQVQLVQSGAEVKKPGASVKVSCKAFGYPFTDYLLHWVRQAPGQGLEWVGWLNPYSGDTNYAQKFQGRVTMTRDTSISTAYMELSRLRSDDTAVYYCTRTTLISVYFDYWGQGTMVTVSSGGSGGSGGSGGSGGSDIQMTQSPSSVSASVGDRVTIACRASQNIRNILNWYQQRPGKAPQLLIYAASNLQSGVPSRFSGSGSGTDFTLTINSLQPEDFATYYCQQSYSMPRTFGGGTKLEIK

SEQ ID NO: 27 - protein, 489 aa, Artificial Sequence
  Synthetic Polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSGSGGSGEVQLVESGGGLVQPGGSLRLSCAASGFTFSRHWMHWLRQVPGKGPVWVSRINGAGTSITYADSVRGRFTISRDNANNTLFLQMNSLRADDTALYFCARANSVWFRGLFDYWGQGTPVTVSSGGSGGSGGSGGSGGSEIVLTQSPVTLSVSPGERVTLSCRASQSVSTNLAWYQQKLGQGPRLLIYGASTRATDIPARFSGSGSETEFTLTISSLQSEDFAVYYCQQYDKWPDTFGQGTKLEIK

SEQ ID NO: 28 - protein, 484 aa, Artificial Sequence
  Synthetic Polypeptide
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKGGGGSGGGGSGGGGSEVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSGSGGSGQVQLVESGGGLVQPGGSLRLSCAASGFTFSSYWMNWVRQAPGKGLEWVSGIENKYAGGATYYAASVKGRFTISRDNSKNTLYLQMNSLRAEDTAVYYCARGFGTDFWGQGTLVTVSSGGSGGSGGSGGSGGSDIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 29 - protein, 159 aa, Artificial Sequence
  Synthetic Polypeptide
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 30 - DNA, 100 bp, Artificial Sequence
  Synthetic Oligonucleotide
gctgcagagcctgctgctctgttttagagctagaaatagcaagttaaaataaggctagtccgttatcaacttgaaaaagtggcaccgagtcggtgctttt

SEQ ID NO: 31 - DNA, 100 bp, Artificial Sequence
  Synthetic Oligonucleotide
ggagcatgtgaatgccatccgttttagagctagaaatagcaagttaaaataaggctagtccgttatcaacttgaaaaagtggcaccgagtcggtgctttt

SEQ ID NO: 32 - DNA, 100 bp, Artificial Sequence
  Synthetic Oligonucleotide
gcatgtgaatgccatccagggttttagagctagaaatagcaagttaaaataaggctagtccgttatcaacttgaaaaagtggcaccgagtcggtgctttt

SEQ ID NO: 33 - DNA, 100 bp, Artificial Sequence
  Synthetic Oligonucleotide
gagacgccgggcctcctggagttttagagctagaaatagcaagttaaaataaggctagtccgttatcaacttgaaaaagtggcaccgagtcggtgctttt

SEQ ID NO: 34 - DNA, 100 bp, Artificial Sequence
  Synthetic Oligonucleotide
gatggcattcacatgctcccgttttagagctagaaatagcaagttaaaataaggctagtccgttatcaacttgaaaaagtggcaccgagtcggtgctttt

SEQ ID NO: 35 - DNA, 100 bp, Artificial Sequence
  Synthetic Oligonucleotide
gctcccagggctgcgtgctggttttagagctagaaatagcaagttaaaataaggctagtccgttatcaacttgaaaaagtggcaccgagtcggtgctttt

SEQ ID NO: 36 - DNA, 100 bp, Artificial Sequence
  Synthetic Oligonucleotide
gcgtgctggggctgggcgaggttttagagctagaaatagcaagttaaaataaggctagtccgttatcaacttgaaaaagtggcaccgagtcggtgctttt

SEQ ID NO: 37 - DNA, 100 bp, Artificial Sequence
  Synthetic Oligonucleotide
gctggggctgggcgagcggggttttagagctagaaatagcaagttaaaataaggctagtccgttatcaacttgaaaaagtggcaccgagtcggtgctttt

SEQ ID NO: 38 - DNA, 100 bp, Artificial Sequence
  Synthetic Oligonucleotide
gacttagtgcaatgcaagacgttttagagctagaaatagcaagttaaaataaggctagtccgttatcaacttgaaaaagtggcaccgagtcggtgctttt

SEQ ID NO: 39 - DNA, 100 bp, Artificial Sequence
  Synthetic Oligonucleotide
gatttacagatgattttgaagttttagagctagaaatagcaagttaaaataaggctagtccgttatcaacttgaaaaagtggcaccgagtcggtgctttt

SEQ ID NO: 40 - DNA, 99 bp, Artificial Sequence
  Synthetic Oligonucleotide
aagaaaacacagctacaacgttttagagctagaaatagcaagttaaaataaggctagtccgttatcaacttgaaaaagtggcaccgagtcggtgctttt

SEQ ID NO: 41 - DNA, 99 bp, Artificial Sequence
  Synthetic Oligonucleotide
caactggagcatttactgcgttttagagctagaaatagcaagttaaaataaggctagtccgttatcaacttgaaaaagtggcaccgagtcggtgctttt

SEQ ID NO: 42 - DNA, 99 bp, Artificial Sequence
  Synthetic Oligonucleotide
tctttgtagaacttgaagtgttttagagctagaaatagcaagttaaaataaggctagtccgttatcaacttgaaaaagtggcaccgagtcggtgctttt

SEQ ID NO: 43 - DNA, 100 bp, Artificial Sequence
  Synthetic Oligonucleotide
gagcactgaaagcatgatccgttttagagctagaaatagcaagttaaaataaggctagtccgttatcaacttgaaaaagtggcaccgagtcggtgctttt

SEQ ID NO: 44 - DNA, 100 bp, Artificial Sequence
  Synthetic Oligonucleotide
ggacgtggagctggccgagggttttagagctagaaatagcaagttaaaataaggctagtccgttatcaacttgaaaaagtggcaccgagtcggtgctttt

SEQ ID NO: 45 - DNA, 100 bp, Artificial Sequence
  Synthetic Oligonucleotide
gaggcgctccccaagaagacgttttagagctagaaatagcaagttaaaataaggctagtccgttatcaacttgaaaaagtggcaccgagtcggtgctttt

SEQ ID NO: 46 - DNA, 100 bp, Artificial Sequence
  Synthetic Oligonucleotide
gggggccccagggctccagggttttagagctagaaatagcaagttaaaataaggctagtccgttatcaacttgaaaaagtggcaccgagtcggtgctttt

SEQ ID NO: 47 - DNA, 100 bp, Artificial Sequence
  Synthetic Oligonucleotide
gctgaggaacaagcaccgccgttttagagctagaaatagcaagttaaaataaggctagtccgttatcaacttgaaaaagtggcaccgagtcggtgctttt

SEQ ID NO: 48 - DNA, 100 bp, Artificial Sequence
  Synthetic Oligonucleotide
ggcgcctgccacgatcaggagttttagagctagaaatagcaagttaaaataaggctagtccgttatcaacttgaaaaagtggcaccgagtcggtgctttt

SEQ ID NO: 49 - DNA, 100 bp, Artificial Sequence
  Synthetic Oligonucleotide
gtgcagcaggcagaagagcggttttagagctagaaatagcaagttaaaataaggctagtccgttatcaacttgaaaaagtggcaccgagtcggtgctttt

SEQ ID NO: 50 - DNA, 99 bp, Artificial Sequence
  Synthetic Oligonucleotide
ggagtgatcggcccccagagttttagagctagaaatagcaagttaaaataaggctagtccgttatcaacttgaaaaagtggcaccgagtcggtgctttt

SEQ ID NO: 51 - protein, 21 aa, Artificial Sequence
  Synthetic Polypeptide
MALPVTALLLPLALLLHAARP

SEQ ID NO: 52 - protein, 242 aa, Artificial Sequence
  Synthetic Polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 53 - protein, 45 aa, Artificial Sequence
  Synthetic Polypeptide
TTTPAPRPPTPAPTIASQPLSLRPEACRPAAGGAVHTRGLDFACD

SEQ ID NO: 54 - protein, 24 aa, Artificial Sequence
  Synthetic Polypeptide
IYIWAPLAGTCGVLLLSLVITLYC

SEQ ID NO: 55 - protein, 42 aa, Artificial Sequence
  Synthetic Polypeptide
KRGRKKLLYIFKQPFMRPVQTTQEEDGCSCRFPEEEEGGCEL

SEQ ID NO: 56 - protein, 112 aa, Artificial Sequence
  Synthetic Polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - protein, 246 aa, Artificial Sequence
  Synthetic Polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 58 - protein, 152 aa, Artificial Sequence
  Synthetic Polypeptide
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 59 - protein, 26 aa, Artificial Sequence
  Synthetic Polypeptide
MATGSRTSLLLAFGLLCLPWLQEGSA

SEQ ID NO: 60 - protein, 178 aa, Artificial Sequence
  Synthetic Polypeptide
MATGSRTSLLLAFGLLCLPWLQEGSARPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE